(19) United States
(12) Patent Application Publication
(10) Pub. No.: US 2026/0232761 A1
(43) Pub. Date: Aug. 13, 2026

(54) USE OF HEMOLYMPH OR HEMOLYMPH COMPONENTS FOR THE TREATMENT AND PREVENTION OF INFLAMMATION AND DRY SKIN

(71) Applicant: Marin Brands Inc., South Portland, ME (US)

(72) Inventors: William Patrick Breeding, South Portland, ME (US); Sadie Elaina Kuhn, Topsham, ME (US); Amber L. Boutiette, South Portland, ME (US)

(73) Assignee: Marin Brands Inc., South Portland, ME (US)

(21) Appl. No.: 19/301,589

(22) Filed: Aug. 15, 2025

Related U.S. Application Data
(63) Continuation-in-part of application No. 17/121,292, filed on Dec. 14, 2020, now Pat. No. 12,403,162.
(63) Continuation-in-part of application No. 17/466,139, filed on Sep. 3, 2021, now Pat. No. 12,433,919.
(60) Provisional application No. 62/948,240, filed on Dec. 14, 2019.
(60) Provisional application No. 63/074,089, filed on Sep. 3, 2020.

(51) Int. Cl. A61K 38/17; A61K 9/00; A61K 9/06; A61K 35/612; A61K 47/06; A61K 47/10; A61K 47/12; A61K 47/14; A61K 47/22; A61K 47/24; A61K 47/34; A61K 47/36; A61K 47/44; A61P 17/00

(52) U.S. Cl. CPC A61K 38/1767; A61K 9/0014; A61K 9/06; A61K 47/06; A61K 47/10; A61K 47/12; A61K 47/14; A61K 47/22; A61K 47/24; A61K 47/34; A61K 47/36; A61K 47/44; A61P 17/00; A61K 35/612

(57) ABSTRACT

Uses are described for hemolymph, contents of hemolymph, fragments or components of the contents found within hemolymph, including hemocyanin, any of which may be derived from crustaceans, as functional agents to treat diseases, provide symptomatic relief, provide a beautifying or relieving effect to the intended organ or site-of-intended-use, or be used as an ingredient in formulations designed to aid in the relief of pathologies or states commonly associated with inflammation and dry skin, including, but not limited to eczema, dermatitis, psoriasis, cystitis, asthma, sinusitis, joint pain, as well as other instances of acute and chronic inflammation, and for relief of general symptoms that may be associated with inflammation and dry skin but not linked directly in the intended use, including, but not limited to, redness, itchiness, irritation, muscle or joint fatigue, swelling, hydrating the skin, and reducing transepidermal water loss (TEWL).

This application is a Continuation-in-Part of U.S. patent application Ser. No. 17/121,292 filed on Dec. 14, 2020, which claims the benefit of U.S. Provisional Application No. 62/948,240, filed on Dec. 14, 2019, and is a Continuation-in-Part of U.S. patent application Ser. No. 17/466,139 filed on Sep. 3, 2021, which claims the benefit of U.S. Provisional Application No. 63/074,089, filed on Sep. 3, 2020 each of which is hereby incorporated by reference herein in its entirety.

### TECHNICAL FIELD

The present invention relates to the use of or administration of hemolymph, or a component of hemolymph, in cosmetics, skincare, supplements, nutrition, pharmaceutical drugs and healthcare, for the general reduction of inflammation, and the hydration or prevention of transepidermal moisture loss from mammalian skin.

### BACKGROUND

The use of naturally-derived compounds in fields such as cosmetics, nutrition and healthcare has been of rising interest. The prospect of new ingredients sourced from marine environments offers a wide range of abundance and possibility for multi-faceted ingredient function. Marine-derived compounds including proteins, peptides, carbohydrates and bacterial strains found in plants and animals have recently seen a significant amount of activity in these fields, with compound sources providing an abundance of availability, clinical studies revealing increasingly more potential to increase mammalian health, and the general populations' attitude toward natural and sustainable products becoming increasingly more favorable.

One untapped source of potential is that of the arthropod and mollusk family, with entire fishing industries dedicated to the catch and sale of meat derived from these sources, which produce immense amount of waste, as components not converted into viable products and sold into market are commonly disposed of in landfills or feed back into the environment. In many cases, the waste-byproducts of the processes offer an array of commercially viable applications beneficial to our society's needs. This potential is often not taken advantage of due to inability of the producer of traditional goods to realize such value-added opportunities, the inability of the producer to implement a viable process to convert these byproducts into value-added products, or the general lack of scientific exploration into such value-added opportunities to understand that they exist. It is believed that one of these untapped sources of potential lies in the use of hemolymph, or components of hemolymph, derived from arthropods or mollusks.

Hemolymph is a natural substance derived from arthropods and mollusks including lobsters, crabs, and oyster species. Certain formulations are known which contain hemolymph, or a glycoprotein found within the hemolymph, called hemocyanin for various uses.

One industry, with waste materials abundant and ripe for innovation, is the lobster industry. The lobster industry brings in millions of pounds of lobster per year in areas around the world, predominantly along North America's east coast—with Maine lobstermen bringing in over 119 million pounds of meat in 2018. In the processing of these lobsters, the meat is the primary component of interest, with other components such as the shell and hemolymph viewed as waste. It is estimated that at this volume of catch, between 1-5 million pounds of hemolymph is wasted, trickling down the drain, each year.

Similar industries involving the growth of catch of marine organisms such as oyster farming in the state of Maine, are growing at promising rates, offering additional streams of hemolymph that have potential, due to variance in biochemical structure, to possess superior strength in applications in comparison to hemolymph, or hemolymph components, from other marine sources. Organisms that are invasive and harmful to marine environments, such as the green crab, also offer streams of hemolymph, which if a strong enough commercial application of the hemolymph existed, could encourage the establishment of processes to collect it, which could yield a high-value product that simultaneously results in the safety and preservation of marine environments. Thus, it is clear that hemolymph from such sources should be explored and value-added applications, such as the ones described in this patent, be validated, documented and commercialized.

Skin conditions falling under the umbrella of complications with inflammation, hydration, and dryness afflict an overwhelming majority of the world's population. Whether due to lifestyle, diet, exercise, atmosphere, hormones, stress, genes, topical irritants, allergies or otherwise, conditions ranging from eczema and psoriasis to general issues with skin hydration and inflammation are on the rise. Current modalities suitable to address such conditions from the therapeutic to cosmetic level include ingredients such as topical steroids, which act aggressively to spot-treat skin but often cause further damage, to basic moisturizers and emollients that serve as mildly effective forms of maintenance but not effective, long-term solutions for troubled skin.

Thus, what is needed in the field is an alternative to limited bland ingredients, that does not cause the adverse effect of a harsh ingredient such as a topical steroid, that possesses the multifaceted capabilities to deliver therapeutic, symptomatic relief and cosmetic effects necessary to address conditions that often present as an amalgam of symptoms or complications. By leveraging one or more of the capabilities of hemolymph, or component within the hemolymph, to reduce inflammation and hydrate or reduce transepidermal moisture loss from dry skin, beneficial therapeutic or cosmetic effects may be realized in a mammalian subject.

### BRIEF DESCRIPTION OF THE FIGURES

FIG. 1 shows digital images of target lesions, comparison of baseline to week 4 post test product application for a variety of skin types A) head and neck, B) trunk, C) upper extremities, and D) lower extremities.

FIG. 2 shows a bar graph of percent visual improvement of skin dryness and smoothness at various time points post application from immediately post application, 48 hours post application, 1 week post application, and 4 weeks post application.

FIG. 3 shows a bar graph of mean EASI score at various time points post application from baseline, immediately post application, 48 hours post application, 1 week post application, and 4 weeks post application (p<0.001 for all time points post application).

FIG. 4 shows a bar graph of the mean Investigator's Global Assessment (IGA) score at various time points post application from baseline, immediately post application, 48 hours post application, 1 week post application, and 4 weeks post application.

FIG. 5 shows a bar graph of study participant experience agreement level (percent satisfaction level) at study end, displaying a minimum average reported agreement level of 87.9% and most common level of 93.9% over the study 4-week period.

FIG. 6 shows a bar graph of results from an MTT assay of fibroblast viability percentage versus dosage of various hemocyanin glycoprotein dosant concentrations (weight percent) tested.

FIG. 7 shows a bar graph of results from an MTT fibroblast assay showing viability percentage versus hemocyanin glycoprotein concentration (mg/mL) of various concentrations tested of 0.003 mg/ml, 0.03 mg/ml, 0.3 mg/ml, 1.5 mg/ml, 3 mg/ml, and 6 mg/ml.

FIG. 8 shows a bar graph of results from a Type I collagen assay showing Type-1 C-Peptide (collagen) production (ng/ml) versus various test components of the test buffer and test glycoprotein composition concentration of 0.01%, 0.1%, 1%, 5%, 10% and 20% hemocyanin glycoprotein.

FIG. 9 shows a bar graph of results of a fibroblast Type I C-Peptide (ng/ml) production assay with various Hemocyanin Glycoprotein concentrations of 0.003 mg/ml, 0.03 mg/ml, 1.5 mg/ml, 3 mg/ml, and 6 mg/ml tested.

FIG. 10 shows a bar graph of results from a Hyaluronic Acid Assay of fibroblast HA content (ng/ml) present in various test composition components at various concentrations of 0.01%, 0.1%, 1%, 5%, 10%, and 20% hemocyanin glycoprotein tested.

FIG. 11 shows a bar graph of results of a Hyaluronic Acid Assay showing hyaluronic acid (ng/ml) in fibroblasts versus various hemocyanin glycoprotein concentrations (mg/ml) dose concentrations of 0.003 mg/ml, 0.03 mg/ml, 0.3 mg/ml, 1.5 mg/ml, 3 mg/ml, and 6 mg/ml tested.

FIG. 12 shows a bar graph of production of pro-inflammatory cytokine IL-6 (pg/ml) by HDFa fibroblasts following cultivation with inflammatory stimulation by lipopolysaccharides (LPS) showing anti-inflammatory effect by lobster hemocyanin glycoproteins at various dosages of 0.01 mg/mL, 0.1 mg/mL, 1 mg/mL, 10 mg/mL, 16 mg/ml, and 25 mg/mL and controls.

FIG. 13 shows a bar graph of antioxidant effect of reduced mean level of reactive oxygen species (ROS) on HDFa fibroblasts following exposure to H₂O₂ and cultivation with lobster hemocyanin glycoproteins at various dosages of 0.01 mg/ml, 0.1 mg/ml, 1 mg/ml, 10 mg/ml, 16 mg/ml, and 25 mg/ml and controls.

FIG. 14 shows a bar graph of antioxidant effect of reduced mean level of reactive oxygen species (ROS) on HaCaT keratinocytes following exposure to H₂O₂ and cultivation with lobster hemocyanin glycoproteins at various dosages of 0.01 mg/ml, 0.1 mg/ml, 1 mg/ml, 2 mg/ml, and 4 mg/ml and controls.

### SUMMARY OF THE INVENTION

In one embodiment, the present disclosure provides a novel use of arthropod hemolymph, and components thereof, as an ingredient or the active ingredient independently or mixed in formulations administered by way of parenteral, oral, nasal, ocular, transmucosal, and transdermal routes, to deliver a cosmetic, symptomatic relief, pharmaceutical drug, nutritional or general benefit to mammalian health. The invention relates to preparations, intended uses, formulations, intended outcomes as a result of use, of hemolymph, or components thereof, rendered preferably, but not exclusively, from lobsters, oysters, crabs or mollusks.

This technology provides a chemical composition comprising a marine ingredient with the capabilities of reducing mammalian inflammation, and hydrating mammalian skin, or reducing transepidermal moisture loss, i.e. transepidermal water loss (TEWL), from mammalian skin.

The hemolymph, hemocyanin or moiety found within hemolymph is able to perform as a gentle and effective alternative to existing treatment or cosmetic options due to its multi-faceted mechanisms of action working in unison together to deliver the desired activity relating to the reduction of inflammation and enhancing skin hydration or reducing transepidermal moisture loss properties in any or all therapeutic, symptomatic relief or cosmetic applications. Additionally, as opposed to many common treatment and cosmetic alternatives, the hemolymph, hemocyanin or moiety found within hemolymph is a gentle alternative as it does not cause adverse irritation or create withdrawal symptoms after discontinuing use.

A method for treating or preventing inflammation in a mammal is described, comprising the steps of: (a) providing a composition comprising hemolymph or a moiety found in hemolymph; and (b) administering the composition to the mammal. In a preferred embodiment, the composition is a topical composition administered topically to the skin.

A further method for treating or preventing dry skin in a mammal is described, comprising the steps of: (a) providing a composition comprising hemolymph or a moiety found in hemolymph; and (b) administering the composition to the mammal. In a preferred embodiment, the composition is a topical composition administered topically to the skin.

### DETAILED DESCRIPTION

This invention relates to the use of hemolymph, hemocyanin, or other moieties found within hemolymph from arthropods, mollusks and recombinantly produced versions of such moieties, having the capability of reducing inflammation, hydrating skin or reducing transepidermal moisture loss from skin, used for the purpose of providing therapeutic, symptomatic relief and/or cosmetic effects to a mammal.

This invention further comprises the administration of hemolymph, a moiety found in hemolymph, a fragment or component of said moiety including but not limited to (preferably) the glycoprotein called hemocyanin, or peptides, used for the purpose of providing therapeutic, symptomatic relief, and/or cosmetic effects to a mammal, by leveraging one or more of the capabilities of reducing inflammation, hydrating skin or reducing transepidermal moisture loss from skin.

In one aspect, the development, and developed viable products, using hemolymph and hemolymph components found in the lobster as an ingredient, or the active ingredient, in cosmetic, skincare, supplements, nutritional supplements and products, pharmaceutical drugs and general healthcare applications, is described. Through investigational preliminary laboratory data and anecdotal evidence supporting the feasibility of these applications, it is believed 1-5 million of pounds of hemolymph, and components found within the hemolymph, can be converted into high-value ingredients and products to provide industries such as the lobster industry with a significantly value-added revenue stream.

Hemolymph may be extracted following a variety of procedures. In a preferred embodiment, hemolymph may be obtained from lobsters as mentioned in U.S. Pat. No. 9,078,906 B2, herein incorporated by reference. U.S. Pat. No. 9,078,906 describes use of hemolymph for antiviral and antineoplastic applications.

The form of hemolymph, or component of hemolymph, used in such products, can vary greatly. Numerous components found within the hemolymph have been shown to possess medicinal applications, such as the hemocyanin or peptides, which could give rise to motivation for using the hemolymph in its naturally-derived state, or isolating such components for more specific use.

In certain preferred embodiments, the components within the hemolymph, such as hemocyanin or peptides, may be isolated or purified in some manner. For components such as hemocyanin or peptides, this may occur using an array of typical purification methods, including, but not limited to, a form of chromatography such as affinity, high pressure liquid, ion-exchange or gel-filtration chromatography, dialysis, electrophoresis methods such as gel or two-dimensional electrophoresis, SDS-PAGE, precipitation, ‘salting out’, isoelectric focusing, immunoblotting, centrifugation, ultracentrifugation, enzymatic cleavage or solid-phase extraction.

Hemolymph, or hemolymph components, as derived naturally or from their respective isolation or purification techniques, could be incorporated into a formulation in crude form as-is, or further cleared of debris or contaminants with any known filtration technique, or converted from liquid to powder form using a process such as lyophilization, or combined with an array of ingredients to complement the efficacy, stability or other properties relating to a preferred use as a product.

The method of hemolymph component purification is certainly tailored to the component of interest, and the method may vary depending on intended component use; however, the true novelty does not lie in the isolation or purification process, rather, the preferred use of the component.

The hemolymph, hemocyanin or other moiety found within hemolymph may be administered independently (just the hemolymph, hemocyanin, or said moiety) or in a chemical composition, which may be delivered to a mammal via any mechanism of delivery.

The hemolymph, hemocyanin or other moiety found within hemolymph used may be obtained through natural (collected from an arthropod or mollusk) or recombinant (produced via culture in bioreactor) means. Such sources, from arthropods and mollusks, may include but are not limited to lobsters, crabs, oysters and mollusks, such as lobster species Nephrops novegicus, Homarus gammarus, Panularis elephas and Homarus americanus, as well as oyster species Crassostrea virginica and Ostrea edulis, crab species Carcinus maenas, Portunus pelagicus, Portunus trituberculatus, Callinectes sapidus, Cancer pagurus, Metacarcinus magister and Chionoecetes, crawfish species Procambarus clarkii and Procambarus zonangulus, shrimp species Penaeus monodon, Pandalus borealis, Acetes japonicus and Trachysalambria curvirostris, and mollusk classes gastropoda, bivalvia and cephalopoda.

The hemolymph, hemocyanin or other moiety found within hemolymph may be used as naturally obtained from the natural or recombinant source (un-modified form), an isomer, subunit, component, or fragment of hemocyanin, a functionalized form, a modified form, a glycosylated form, a form resuspended in a physiologically acceptable buffer or lyophilized form, or prepared in any other method.

One preferred way to practice the invention is to incorporate the hemolymph, hemocyanin or moiety found within hemocyanin to a composition as a delivery vehicle for topical administration to mammalian skin.

### Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films or oral dissolvable strips can be used. Other useful delivery systems comprise oral or nasal sprays or inhalers, and the like.

For oral administration, the compounds or extracts may be combined with one or more solid inactive ingredients for the preparation of tablets, capsules, pills, powders, granules or other suitable dosage forms. For example, the active agent may be combined with at least one excipient such as fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like.

Further for oral administration, the compounds or extracts may be incorporated with an excipient and used in the form of ingestible tablets, buccal tablets, troches, capsules, elixirs, suspensions, syrups, wafers, and the like. The tablets, troches, pills, capsules, and the like can also contain the following: a binder such as gum tragacanth, acacia, corn starch, or gelatin; excipients such as dicalcium phosphate; a disintegrating agent such as corn starch, potato starch, alginic acid, and the like; a lubricant such as magnesium stearate; and a sweetening agent such as sucrose, lactose, or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the active compound, sucrose as a sweetening agent, methyl and propylparabens as preservatives, a dye, and flavoring such as cherry or orange flavor. Oil-in-water emulsions may be better suited for oral use in infants because these are water-miscible, and thus their oiliness is masked. Such emulsions are well known in the pharmaceutical sciences.

### Routes of Administration

The compounds or extracts may be administered by any route, including but not limited to oral, sublingual, buccal, ocular, pulmonary, rectal, and parenteral administration, or as an oral or nasal spray (e.g. inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of hemolymph in the body of the patient in a controlled formulation, with systemic or local release of the drug to occur at a later time. For example, the drug may be localized in a depot for controlled release to the circulation, or for release to a local site of a growth or area of concern.

Regardless of application; therapeutically used to address a pathology, to mitigate symptoms, or for cosmetic effects, the composition may be comprised of a number of additional ingredients that provide complementary or supplementary effects, and delivered through any vehicle and method desired. Potential delivery mechanisms include, but are not limited to parenteral, oral, transmucosal, transdermal or topical routes; via intravenous, subcutaneous, intrathecal, intramuscular or site-specific ingestion routes, as a solution, suspension or emulsion, liquid suspension, physical mixture, encapsulated liquid suspension, encapsulated physical mixture, spray, topical liquid, emulsion, suspension, ointment, encapsulation, gelling systems, micelle, dendrimer, liposome, nanoparticle or time delivery system, dendrimers, liposomes, rectally, vaginally, buccally, or applied to the skin as a gel, cream, ointment, powder, emulsion, paste, spray, suspension, foaming agent, serum, shampoo, wax, balm, emollient, oil, lotion, band-aid, patch-test or suspended within an encapsulation.

In an embodiment, the invention relates to a chemical composition comprising at least hemolymph, a moiety found in hemolymph, a fragment or component of said moiety including but not limited to (preferably) hemocyanin or peptides, having the capability of reducing inflammation, hydrating skin or reducing transepidermal moisture loss from skin, used for the purpose of providing therapeutic, symptomatic relief and/or cosmetic effects to a mammal.

The hemolymph or moiety (glycoprotein, hemocyanin) found within hemolymph used may be obtained through natural (collected from an arthropod or mollusk) or recombinant (produced via culture in bioreactor) means, as discussed herein, and may be used in a composition.

In a therapeutic embodiment, the composition may be used to address pathologies relating to inflammation, skin hydration, or loss of skin moisture content, or used for treatment modalities for the purpose of indirectly addressing a pathology. Such pathologies may include general dermatitis, atopic dermatitis, allergic contact dermatitis, seborrheic dermatitis, radiation dermatitis, radiation exposure irritation, xerosis, perioral dermatitis, stasis dermatitis, pregnancy-induced dermatitis, perineal dermatitis, autoimmune disorders, eczema, psoriasis, rosacea, pruritus, acne, rashes, contact burns, cuts, fungal skin infections, bacterial skin infections, vitiligo, inflammatory bowel disease, coeliac disease, alopecia areata, hives, sunburns, photoallergic skin reactions, abnormal scarring keloids, asthma, cystitis, sinusitis, transplant rejection, occupational inflammatory or dry skin conditions, dermopathy, cercarial dermatitis, lichen planus, incision inflammation, irritation or redness care and post-surgical inflammation, redness or irritation incision care.

In a symptomatic relief embodiment, the composition may be used to relieve or modulate symptoms directly or indirectly associated with a host of inflammatory or dry skin conditions as described; symptoms commonly associated with inflammatory conditions, including erythema, papules, erosion, exudation, itching, redness, swelling, fatigue, joint stiffness, joint pain, drug rash, heat rash, intertrigo and diaper rash.

The composition made in accordance with the embodiments herein may also address symptoms commonly associated with abnormalities in skin hydration, including decreasing transepidermal moisture loss, binding moisture, increasing skin moisture content, altering sebum production, and decreasing skin dryness.

A third embodiment involves the use of the chemical composition for delivering a range of cosmetic effects. These cosmetics effects highly overlap with those of the symptom management applications, functions which may be interchanged between symptomatic relief and cosmetic purposes, including soothing, calming, calming aggravated, sensitive, troubled, ‘angry’, ‘upset’ or irritated skin, relieving, redness reducing, helping skin retain moisture, combatting dehydration, or acting as a humectant.

The material used in a composition made in accordance with the embodiments herein may be used as naturally obtained from the natural or recombinant source (un-modified form), an isomer, subunit, component, or fragment of hemocyanin, a functionalized form, a modified form, a glycosylated form, a form resuspended in a physiologically acceptable buffer or lyophilized form, or prepared in any other method.

Regardless of application; therapeutically used to address a pathology, to mitigate symptoms, or for cosmetic effects, the composition may be comprised of a number of additional ingredients that provide complementary or supplementary effects, and delivered through any vehicle and method desired. Potential delivery mechanisms include, but are not limited to Parenteral, Oral, Transmucosal, Transdermal/Topical routes; via intravenous, subcutaneous, intrathecal, intramuscular or site-specific ingestion routes, as a solution, suspension or emulsion, liquid suspension, physical mixture, encapsulated liquid suspension, encapsulated physical mixture, spray, topical liquid, emulsion, suspension, ointment, encapsulation, gelling systems, micelle, dendrimer, liposome, nanoparticle or time delivery system, dendrimers, liposomes, rectally, vaginally, buccally, or applied to the skin as a gel, cream, ointment, powder, emulsion, paste, spray, suspension, foaming agent, serum, shampoo, wax, balm, emollient, oil, lotion, band-aid, patch-test or suspended within an encapsulation.

Hemolymph extracts or fractions are useful for the present invention.

### Process for Preparing Hemocyanin Active Ingredient (Natural Hemocyanin)

In one embodiment, the hemocyanin may be obtained from a natural source (e.g. from arthropods), and prepared in a method, as follows.

The hemolymph of the arthropod is collected via vacuum or gravity feed into a holding container. This can be accomplished in a number of ways; the arthropod or mollusk, preferably lobster, can be lanced in easily penetrable or soft areas, including across the abdomen area adjacent to the swimmerets, allowing for hemolymph to drain, a needle may be inserted to allow for hemolymph to gravimetrically drain or be vacuum drained, the lobster may be placed on an mechanical or stationary lancing and flow system that lances the underbelly and directs hemolymph flow into a basin, the lobster abdomen may be separated from the thorax, as performed in traditional seafood processing, and the resulting hemolymph flow draining out may be directed into a basin.

The preferred methods of hemolymph collection are via vacuum system, using a sterilized 16 gauge needle attached to a tube which feeds into a collection basin, drawing hemolymph at a rate between 80-160 mL/min. Additionally, it is preferred to use an automated or stationary lancing and flow system, where the lobster may be manually lanced by an operator using a needle or sharp edge and placed onto a holder which allow hemolymph to flow and directs said flow into a collection basin, or, the operator may place the lobster on the holder, which employs a stationary or mechanically actuated lancing needle or sharp edge, thus allowing for full automation of the lancing and hemolymph flow into basin process. Finally, a third preferred method of hemolymph collection simply involves the use of a needle attached to a temporary collection vessel, such as a syringe or tube attached to a bulb, to draw the hemolymph from the lobster and subsequently deposit the hemolymph into a storage basin.

The collected hemolymph can be immediately stored in a −20° C. freezer (to be later thawed and subjected to the following step), allowed to sit in ambient conditions for no longer than two hours, in which the hemolymph clotting cascade naturally forms layers of clotted cellular mass/debris and a hemocyanin rich liquid layer, or the hemolymph is immediately subjected to chemical or physical perturbation to further activate the clotting cascade and expedite the formation of the solid and liquid layers.

Once the solid and liquid layers are formed, they may then be separated via centrifugation and decanting the liquid layer, filtration (vacuum, flow, gravimetric through a membrane, porous material, filter, etc.) to isolate the liquid layer, siphoning, draining, or another form of separation to remove the solid layer and isolate the liquid, hemocyanin-rich layer.

Preferably, the solid layer is filtered out by gravimetric or vacuum flow through a 0.22-40 μm filter, allowing the filter to remove the large solid particulates in solid phase, resulting in a clear, hemocyanin-rich liquid phase.

An additional preferable method to isolate the hemocyanin-rich liquid phase may be to centrifuge the hemolymph at between 1,000 g-12,000 g for between 5-60 minutes at a temperature between 4-20° C., and decant the supernatant to obtain the hemocyanin-rich liquid layer.

The concentration of hemocyanin suspended in the resulting hemocyanin-rich liquid layer may be of interest and can be assessed by any normal means of measuring protein concentration. Preferably, absorbance, refractometry and the Bradford assay are used to measure protein concentration. Hemocyanin, regardless of the source, displays absorbance bands at 280 nm and 350 nm. If absorbance is used, the 280 nm is the most accurate and reliable measure of concentration, as the 350 nm peak may vary depending on the degree of oxygenation of hemocyanin in solution. Therefore, more preferably, refractometry may be used to assess hemocyanin concentration by way of estimation of total protein content. Hemocyanin, depending on the source, accounts for between 90-99.9% of the total protein content, therefore refractometry is a simple high-level means of assessment. Finally, the Bradford assay may be used to measure protein content, following a standard procedure to measure concentration.

With the hemocyanin-rich layer isolated, a number of steps may be performed based on the required degree of standardization and purification of the end ingredient used in a composition.

In one embodiment, the hemocyanin-rich liquid layer may be used as obtained from the filtration step, where the protein content of the hemocyanin-liquid layer obtained from filtration is generally 90-99.9% hemocyanin.

In another embodiment, the hemocyanin-rich liquid may be subjected to lyophilization to obtain a dried form of the solution components, primarily being hemocyanin. One may elect to add a sugar to the hemocyanin-rich liquid to enhance protein thermal stability, thus decreasing the likeliness of denaturing and unfolding during the freezing and dehydration process. Preferably, sucrose or trehalose are added to the solution, at an amount of 10-800% 10-800% w/w sugar to hemocyanin, ideally 20-80% sucrose w/w hemocyanin and 50-600% trehalose w/w hemocyanin. However, it is possible for the hemocyanin-rich layer to be lyophilized without the addition of a sugar. The solution should be lyophilized for at least 8 hours and can be performed at a range of temperatures preferably between −80° C. and −50° C. (Zanjani, et al., Formulation of abalone hemocyanin with high antiviral activity and stability, European Journal of Pharmaceutical Sciences (2014) 53(1), 77-85. https://doi.org/10.1016/j.ejps.2013.11.013).

In a third embodiment, the hemocyanin-rich liquid layer may be diluted based on the concentration of hemocyanin in solution, to meet a concentration standard set by those preparing the end composition. In an example, the hemocyanin-rich liquid layer may be diluted in distilled water, saline, PBS or another solution commonly used in cosmetics and biotechnology, preferably a physiologically acceptable, non-toxic, non-irritating solution.

In a fourth embodiment, the hemocyanin may be further purified to remove or isolate the protein from the liquid layer. To do so, a number of physical and chemical separation methods may be employed to remove the protein from solution, including centrifugation, ultracentrifugation, precipitation, filtration, chromatography or any other physical or chemical method to isolate the protein from solution.

Precipitation, also referred to as ‘salting out’ is one preferred method of hemocyanin purification, as the method, especially considering the utilization of ammonium sulfate, is ideal for use in scaling up and large-scale purification, being relatively simple, cost-effective, and allowing for large, continuous quantities of raw material to be purified. While an array of salts may be used, ammonium sulfate is preferred for a precipitation procedure. Following a simple method described by Nichols et al., and well known in the field of protein purification, ammonium sulphate crystals can be continuously added to hemocyanin-rich solution and stirred over a period of 12 hours, at 4 C, to reach 50% ammonium sulphate saturation, followed by centrifuging the solution at 10,000 rpm at 4° C. for one hour, decanting, resuspending the pellet and repeated 2-3 times to ensure purity. Following this, the solution was dialyzed against a suitable stabilizing buffer to remove ammonium sulfate and obtain hemocyanin in solution, and finally filtered with a 0.22-0.4 μm filter to sterilize (K. Idakieva, et al. Purification of Hemocyanin from Marine Gastropod Rapana Thomasiana using Ammonium Sulfate Precipitation Method, Biotechnology & Biotechnological Equipment (2009) 23:3, 1364-1367, DOI: 10.1080/13102818.2009.10817671).

Ultracentrifugation may also be used to isolate the hemocyanin from the hemocyanin-rich liquid, following a simple procedure of centrifuging the solution between 20,000-80,000 rpm, preferably between 30,000-50,000 rpm, at 5° C. for a timespan between 1-18 hours (Guo, D., et al. Functional properties of hemocyanin from Oncomelania hupensis, the intermediate host of Schistosoma japonicum. Experimental Parasitology (2009) 123(3), 277-281; Keller, H., et al. Abalone (Haliotis tuberculata) hemocyanin type 1 (HtH1), Eur. J. Biochem. (1999) 264: 27-38).

While precipitation may be an ideal method to isolate the hemocyanin from an economic, scale and simplicity standpoint, the hemocyanin may be purified through a number of different means, including SDS-PAGE, gel or affinity chromatography, HPLC and other forms of protein purification, all of which have been fairly explored in the isolation of hemocyanin from various crustaceans by numerous research groups, and can be performed using standard procedures by those well versed in the art of protein purification.

Regardless of the purification step, once the hemocyanin is isolated (if such a series of steps is desired for use), it may be resuspended in another solution commonly used in cosmetics and biotechnology, including saline, PBS, or preferably another physiologically acceptable, non-toxic, non-irritating solution. Additionally, the hemocyanin may be subjected to lyophilization following the previously mentioned lyophilization procedure obtain a dried, solid form.

The purity of the resulting hemocyanin may be assessed using SDS-PAGE, gel chromatography, TEM, Western Blot, absorption spectroscopy and other analytical techniques commonly practiced in the field of protein purification and characterization, following standard procedures. In chromatography procedures, as hemocyanin is most abundant in the lobster as a ~75 kDa protein, it is recommended that standards such as ovalbumin, beta-galactosidase and ovotransferrin are used, having similar weights to the hemocyanin protein and thus allowing for close and accurate standards to compare to. For absorbance spectroscopy, as previously mentioned, 280 nm should be used to assess the concentration of hemocyanin, referencing an equation for concentration obtained by plotting Bradford assay concentration measurements against absorbance and relating the two measurements to a known concentration.

Additional steps may be employed to modulate or enhance the desired activity or efficacy of the hemocyanin, or hemocyanin-rich liquid, including obtaining a certain isoform, glycoform or subunit assemblies. As previously mentioned, sugars such as sucrose and trehalose may be added to the hemocyanin-rich liquid, or a solution of purified resuspended hemocyanin, to further glycosylate the protein, which may be advantageous both for enhanced thermal stability as well as enhanced efficacy in use cases including the reduction of inflammation, enhancement of hydration and prevention of transepidermal moisture loss properties.

In some cases, it may be of interest to isolate and utilize specific subunit arrangements of hemocyanin, in which a standard method to isolate the subunit may be followed, preferably using affinity chromatography or pH modulation as the simplest means to obtain either a single subunit, or the desired subunit aggregation state. The single lobster hemocyanin subunit is known to weigh roughly 75 kDa, often arranging as a monomer, and as hexamers and dodecamers.

Molon et al. showed how raising a hemocyanin-containing solutions' pH to 9 resulted in nearly 100% dissociation of hemocyanin aggregation states, therefore obtaining a solution of nearly 100% hemocyanin monomers (Molon, et al., Molecular heterogeneity of the hemocyanin isolated from the king crab Paralithodes camtschaticae, Eur. J. Biochem. (2000) 267: 7046-7057). Correspondingly, by lowering the pH and adding Ca²⁺ resulted in the reassociation of the subunits into dodecamer and hexamer forms. Interestingly, Pan et al. demonstrated how between the dodecamer and hexamer, the dodecamer is primarily found in freshly pooled hemolymph, however, upon storing at 4° C. for 3 days, the hexamer became the main form, which evidently was more stable, suggesting that temperature may also be modulated to obtain the desired subunit aggregation state (Pan, J. Y., et al., Dodecamer is required for agglutination of Litopenaeus vannamei hemocyanin with bacterial cells and red blood cells, Marine Biotechnology (2008) 10(6), 645-652. https://doi.org/10.1007/s10126-008-9115-8).

It has been shown that hemocyanins that exhibit impressive polymorphism (in other arthropods) result in protein isoforms that better suited for certain healthcare or biotechnology applications. Additionally, numerous studies have linked hemocyanin glycoform, the specific type or extent of the protein glycosylation, to efficacy on a broad spectrum from pathology treatment to altering cosmetic appearances, which is of specific interest for cosmetic and dry skin applications, where glycoprotein degree of glycosylation plays a major factor in skin hydrating activity. Finally, the hemocyanin subunit arrangement from monomer to hexamer and dodecamers may result in altered efficacy depending on the specific use case and mode of delivery, therefore altering the pH or utilizing another separative technique to isolate the clusters of subunits in varying arrangements may be advantageous.

Therefore in this invention, what may be used as the ‘active’ or ‘key’ ingredient includes a hemocyanin-rich liquid obtained as naturally obtained from hemolymph, a hemocyanin-rich liquid diluted to a desired concentration, a hemocyanin-rich liquid containing the desired hemocyanin isoform, glycoform or subunit aggregation state, a purified hemocyanin isolated from the liquid in dried form or resuspended in a new solution, or a purified hemocyanin with desired isoform, glycoform or subunit aggregation state.

### Process for Preparing Hemocyanin Active Ingredient (Recombinant Hemocyanin)

In another embodiment, the hemocyanin used for the described applications may be recombinantly produced via large scale bioreactor fermentation. The recombinant protein or peptide, or portions thereof, may share at least 85% sequence identity compared to wild-type hemocyanin of arthropods or mollusks. Alternatively, the recombinant protein or peptide, or portions thereof, may share at least 90% sequence identity, or 91%, 92%, 93%, 94%, 95%, 96%, 97%, 98%, 99%, or greater, sequence identity compared to wild-type hemocyanin of arthropods or mollusks.

This can be accomplished following a standard recombinant protein production procedure. First, one must understand the DNA sequence that codes for the hemocyanin, or hemocyanin subunit of interest, and obtain the RNA from the original arthropod or mollusk source. A fragment can be cut and inserted into a suitable vector. This vector is introduced into a suitable host system (in vitro, E. Coli, Yeast, Mammalian Cell), and the system is transformed to express the DNA encoding for the desired hemocyanin or hemocyanin subunit. Following small scale tests to ensure the correct protein is expressed (measurable by simple and standard assays including ELISA and Western Blots) in the desired range of yield, production can be scaled up to fermentation in a large bioreactor.

Glycoproteins can be purified from solution by lysis of culture cells, centrifuging the solution and decanting the supernatant containing the glycoprotein. Presence, purity, concentration and other relevant information pertaining to the produced glycoprotein can analyzed using SDS-PAGE, gel chromatography, TEM, Western Blot, absorption spectroscopy and other analytical techniques commonly practiced in the field of protein purification and characterization.

Following the collection and purification of the resulting protein, again, additional steps may be taken to result in optimal isoforms or optimally glycosylated proteins, depending on the desired use case for the protein. A preferable modification to the resulting protein is glycosylation, which is more conveniently accomplished post-production as opposed to expression of the protein in a glycosylated form, or glycosylation in the reactor, where glycosylated forms of glycoproteins and hemocyanins have shown to possess enhanced efficacy related to various biomedical and cosmetic applications.

Thus, in these embodiments, the hemocyanin material used is a recombinant hemocyanin, or hemocyanin subunit, which may be further treated to shift the proteins towards a desired isoform, subunit assembly, or degree of glycosylation. This hemocyanin or hemocyanin subunit may be used as an active material that is suspended or resuspended in a desired solution or lyophilized and supplied in dried, powder form. In a preferred embodiment, a hemolymph containing formulation or composition may contain hemocyanin in a dosage or amount ranging from about 1.2 ng to about 472 mg.

The intended uses described herein range from cosmetic and nutritional products and supplements to products for mitigation of symptoms and pharmaceutical drugs, without limitation. Provided below are examples of patient diary data anecdotally documenting the result of using a topical formula containing hemolymph, for enhancing visual appearance and feeling of skin, and self-assessed mitigation of symptoms commonly associated with their self-assessed pathology.

In an embodiment, a hemolymph containing formulation may contain from about 0.05% by weight hemolymph or a moiety found in hemolymph, to about 20% by weight hemolymph or a moiety found in hemolymph, based on the total weight of the formulation. In an alternative embodiment, the hemocyanin (e.g. marine glycoprotein), can be used in the range of 0.0052-25% by weight in the above formulation.

Following are several initial test examples.

### Eczema

Case 1: A young adult female with a manifestation of eczema applied to her flared-up facial areas a topical formulation comprised of 20% hemolymph by weight, hyaluronic acid, aloe barbadensis juice, prunus dulcis oil, emulsifying wax, stearic acid, glycerin, phenoxy-ethanol, tocopherol, Persea americana oil, Simmondsia chinensis oil, sheabutter, xanthan gum, lecithin, salix alba extract, azadirachta indica oil, carbomer, triethanolamine, Rosmarinus officinalis oleoresin, tetrasodium EDTA and citric acid. She spot-treated the local eczema flare-ups numerous times throughout a typical day, as-needed, applying a dollop of formulation, effectively 1.25 grams of hemolymph per application, for a time period of 7 days. After this period, she experienced clearing of her eczema afflicted areas and associated symptoms of dryness, redness, itching, and inflammation.

Case 2: An middle-aged adult female with a manifestation of eczema applied to her flared-up hands a topical formulation comprised of 20% hemolymph by weight, hyaluronic acid, aloe barbadensis juice, prunus dulcis oil, emulsifying wax, stearic acid, glycerin, phenoxy-ethanol, tocopherol, Persea americana oil, Simmondsia chinensis oil, sheabutter, xanthan gum, lecithin, salix alba extract, azadirachta indica oil, carbomer, triethanolamine, Rosmarinus officinalis oleoresin, tetrasodium EDTA and citric acid. She spot-treated the local eczema flare-ups numerous times throughout a typical day, as-needed, applying a dollop of formulation, effectively 1.25 grams of hemolymph per application, for a time period of 4 days. After this period, she experienced clearing of her eczema afflicted areas and associated symptoms of dryness, redness, itching, and inflammation.

Case 3: A young adult female with a manifestation of eczema applied to her flared-up face and arms a topical formulation comprised of 20% hemolymph by weight, hyaluronic acid, aloe barbadensis juice, prunus dulcis oil, emulsifying wax, stearic acid, glycerin, phenoxy-ethanol, tocopherol, Persea americana oil, Simmondsia chinensis oil, sheabutter, xanthan gum, lecithin, salix alba extract, azadirachta indica oil, carbomer, triethanolamine, Rosmarinus officinalis oleoresin, tetrasodium EDTA and citric acid. She spot-treated the local eczema flare-ups numerous times throughout a typical day, as-needed, applying a dollop of formulation, effectively 1.25 grams of hemolymph per application, for a time period of 7 days. After this period, she experienced clearing of her eczema afflicted areas and associated symptoms of dryness, redness, itching and inflammation.

Case 4: A young male with a manifestation of eczema on his arms, legs and neck applied a topical formulation comprised of 20% hemolymph by weight, hyaluronic acid, aloe barbadensis juice, prunus dulcis oil, emulsifying wax, stearic acid, glycerin, phenoxy-ethanol, tocopherol, Persea americana oil, Simmondsia chinensis oil, sheabutter, xanthan gum, lecithin, salix alba extract, azadirachta indica oil, carbomer, triethanolamine, Rosmarinus officinalis oleoresin, tetrasodium EDTA and citric acid. He spot-treated the local eczema flare-ups numerous times throughout a typical day, as-needed, applying a dollop of formulation, effectively 1.25 grams of hemolymph per application, for a time period of 4 days. After this period, he no longer experienced symptoms of dryness, redness, itching and inflammation.

### Psoriasis

Case 1: An middle-aged adult female with a manifestation of psoriasis applied to her patch of psoriasis a topical formulation comprised of 20% hemolymph by weight, hyaluronic acid, aloe barbadensis juice, prunus dulcis oil, emulsifying wax, stearic acid, glycerin, phenoxy-ethanol, tocopherol, Persea americana oil, Simmondsia chinensis oil, sheabutter, xanthan gum, lecithin, salix alba extract, azadirachta indica oil, carbomer, triethanolamine, Rosmarinus officinalis oleoresin, tetrasodium EDTA and citric acid. She spot-treated the local patch of psoriasis numerous times throughout a typical day, as-needed, applying a dollop of formulation, effectively 1.25 grams of hemolymph per application, for a time period of 3 days. After this period, she experienced clearing of her psoriatic area and associated symptoms of dryness, redness, inflammation and itching.

### Acne

Case 1: A young adult female with mild to moderate acne applied to her acne a topical formulation comprised of 20% hemolymph by weight, hyaluronic acid, aloe barbadensis juice, prunus dulcis oil, emulsifying wax, stearic acid, glycerin, phenoxy-ethanol, tocopherol, Persea americana oil, Simmondsia chinensis oil, sheabutter, xanthan gum, lecithin, salix alba extract, azadirachta indica oil, carbomer, triethanolamine, Rosmarinus officinalis oleoresin, tetrasodium EDTA and citric acid. She spot-treated the local eczema flare-ups numerous times throughout a typical day, as-needed, applying a dollop of formulation, effectively 1.25 grams of hemolymph per application, for a time period of 2 days. After this period, she experienced a reduction of redness and inflammation.

In a preferred embodiment, methods of the invention to treat atopic dermatitis comprise applying to the affected area topical compositions comprising therapeutically effective amounts of lobster hemocyanin glycoprotein active ingredient compositions in concentrations ranging from greater than about 1% to about 20% by weight, more preferably about 10% to about 20% by weight. In a preferred embodiment, the therapeutically effective amount of lobster hemocyanin active ingredient in the topical composition is present in a concentration ranging from about 6 mg/mL to about 10 mg/mL. In a preferred embodiment, the therapeutically effective amount of lobster hemocyanin active ingredient in the topical composition is present in a mass dosage ranging from about 3 mg to about 30 mg.

In a preferred embodiment, methods of the invention to treat skin conditions by promoting fibroblast cell viability and proliferation, promote Type I Collagen synthesis, and promote hyaluronic acid production comprise applying to the affected area topical compositions comprising therapeutically effective amounts of lobster hemocyanin glycoprotein active ingredient compositions in concentrations ranging from greater than about 1% to about 20% by weight. In a preferred embodiment, the therapeutically effective amount of lobster hemocyanin active ingredient in the topical composition is present in a concentration ranging from about 0.2 mg/mL to about 6 mg/mL. In a preferred embodiment, the therapeutically effective amount of lobster hemocyanin active ingredient in the topical composition is present in a mass dosage ranging from about 0.1 mg to about 3 mg.

In a preferred embodiment, methods of the invention to treat skin inflammation and photoaging comprise applying to the affected area topical compositions comprising therapeutically effective amounts of lobster hemocyanin glycoprotein active ingredient compositions in concentrations ranging from greater than about 1% to about 20% by weight. In a preferred embodiment, the therapeutically effective amount of lobster hemocyanin active ingredient in the topical composition is present in a concentration ranging from about 0.1 mg/mL to about 25 mg/mL. In a preferred embodiment, the therapeutically effective amount of lobster hemocyanin active ingredient in the topical composition is present in a mass dosage ranging from about 0.1 mg to about 25 mg.

According to an embodiment of the present invention, the compositions of the invention contain hemolymph and components thereof, particularly hemocyanin, as active ingredients. The hemolymph or hemocyanin may be present in a therapeutically effective amount, from about 1.0% to about 20.0% by weight of the total weight of the dosage form. In certain embodiments, the therapeutically effective amount of the active hemocyanin ingredient in each dosage form is about 1.0% to about 25% by weight of the total weight of the dosage form, e.g., 1%, 2%, 3%, 4%, 5%, 6%, 7%, 8%, 9%, or 10%, 11%, 12%, 13%, 14%, 15%, 16%, 17%, 18%, 19%, 20%, 21%, 22%, 23%, 24%, 25%. In certain embodiments, the therapeutically effective amount of the active ingredient in each dosage form is about 25 ng to about 472 mg, in another embodiment the therapeutically effective amount of the active ingredient in each dosage form is about 1 ng to about 500 mg, specifically about 1 ng to about 25 ng, about 25 ng to about 50 ng, about 50 ng to about 100 ng, about 100 ng to about 200 ng, about 200 ng to about 500 ng, about 500 ng to about 1 μg, about 1 μg to about 100 μg, about 100 μg to about 500 μg, about 500 μg to about 1 mg, about 1 mg to about 10 mg, e.g., 1 mg, 2 mg, 3 mg, 4 mg, 5 mg, 6 mg, 7 mg, 8 mg, 9 mg or 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, and 500 mg±5 mg. In certain embodiments, the therapeutically effective amount of the active ingredient in each dosage form is about 1 mg to about 10 mg, e.g. 1 mg, 2 mg, 3 mg, 4 mg, 5 mg, 6 mg, 7 mg, 8 mg, 9 mg or 10 mg. Hemocyanin analogs which substantially imitate the function of hemocyanin on the human body can be used in place of hemocyanin in the formulations and methods of the present invention.

### Example 1

### Eczema

59 individuals with eczema, including Asian, African American, Hispanic, and White ethnicities, from toddler to 65+ age groups, topically applied a cream composition containing hemocyanin to areas of the body affected by an eczema flare up, including the Face, Neck, Scalp, Nose, Eyelid, Perioral, Armpit, Upper arm, Elbow, Forearm, Hand, Finger Stomach, Chest, Lower and Upper Back, Thigh, Shin, Knee, Feet areas. The composition contained Water (Aqua), Hemocyanin (Marine Glycoprotein), Butyrospermum Parkii Shea Butter Fruit, Cetyl Alcohol, Glyceryl Stearate, Glycerin, Caprylic/Capric Triglyceride, Sorbitan Oleate Decylglucoside Crosspolymer, Polyacrylamide, C13-14 Isoparaffin, Laureth-7, Cocos Nucifera (Coconut) Oil, Squalane, Tocopherol (Vitamin E), Hyaluronic Acid, Sodium Benzoate, Sodium Levulinate, Potassium Sorbate, and Sodium Phytate. User experience was assessed by subjective participant self-reporting, and responses were collected at times users were able to confidently self-report significant results, ranging between 1-7 days of cream usage. Of this group, 91% reported the cream felt soothing on the skin, significantly reduced itching, left the skin feeling significantly hydrated, visibly reduced dryness, visibly reduced the appearance of a flare up, visibly reduced redness, visibly reduced inflammation, and left the skin appearing visibly more healthy.

### Example 2

### Psoriasis

15 individuals with psoriasis, including Asian, African American, Hispanic, and White ethnicities, from toddler to 65+ age groups, topically applied a cream composition containing hemocyanin to areas of the body affected by psoriasis, including the Face, Scalp, Eyelid, Nose, Hand, Thigh, Knee. The composition contained Water (Aqua), Hemocyanin (Marine Glycoprotein), Butyrospermum Parkii Shea Butter Fruit, Cetyl Alcohol, Glyceryl Stearate, Glycerin, Caprylic/Capric Triglyceride, Sorbitan Oleate Decylglucoside Crosspolymer, Polyacrylamide, C13-14 Isoparaffin, Laureth-7, Cocos Nucifera (Coconut) Oil, Squalane, Tocopherol (Vitamin E), Hyaluronic Acid, Sodium Benzoate, Sodium Levulinate, Potassium Sorbate, and Sodium Phytate. User experience was assessed by subjective participant self-reporting, and responses were collected at times users were able to confidently self-report significant results, ranging between 1-7 days of cream usage. Of this group, 93% reported the cream felt soothing on the skin, significantly reduced itching, left the skin feeling significantly hydrated, visibly reduced dryness, visibly reduced redness, visibly reduced inflammation, visibly reduced the appearance of a flare up and left the skin appearing visibly more healthy.

### Example 3

### Dry, Aggravated Skin

47 individuals self-reporting dry, aggravated skin, including Asian, African American, Hispanic, and White ethnicities, from toddler to 65+ age groups, topically applied a cream composition containing hemocyanin to areas of the body exhibiting dry, aggravated skin, including the Face, Neck, Chest, Forearm, Hand, Finger, Thigh, Knee, Shin, Feet areas. User experience was assessed by subjective participant self-reporting, and responses were collected at times users were able to confidently self-report significant results, ranging between 1-7 days of cream usage. The composition contained Water (Aqua), Hemocyanin (Marine Glycoprotein), Butyrospermum Parkii Shea Butter Fruit, Cetyl Alcohol, Glyceryl Stearate, Glycerin, Caprylic/Capric Triglyceride, Sorbitan Oleate Decylglucoside Crosspolymer, Polyacrylamide, C13-14 Isoparaffin, Laureth-7, Cocos Nucifera (Coconut) Oil, Squalane, Tocopherol (Vitamin E), Hyaluronic Acid, Sodium Benzoate, Sodium Levulinate, Potassium Sorbate, and Sodium Phytate. Of this group, 91% reported the cream felt soothing and calming on the skin, significantly reduced itching and discomfort, left the skin feeling significantly hydrated, visibly reduced redness and dryness, calmed hands aggravated from excessive hand washing and sanitizing, mitigated dryness and inflammation from wind burn, wind rash, temperature and weather-induced dryness, and redness, visibly reduced inflammation, and left the skin appearing visibly more healthy.

### Example 4

### Rashes

8 individuals with rashes including poison ivy, brown tail moth rash and diaper rash, including White and Hispanic ethnicities, ranging from toddler to 65+ age groups, topically applied a cream composition containing hemocyanin to areas of the body affected by an itchy, inflamed rash, including the Face, Scalp, Neck, Hand, Finger, Forearm, Chest, Stomach, Back, Upper arm, Thigh, Knee, Shin and Feet areas. The composition contained Water (Aqua), Hemocyanin (Marine Glycoprotein), Butyrospermum Parkii Shea Butter Fruit, Cetyl Alcohol, Glyceryl Stearate, Glycerin, Caprylic/Capric Triglyceride, Sorbitan Oleate Decylglucoside Crosspolymer, Polyacrylamide, C13-14 Isoparaffin, Laureth-7, Cocos Nucifera (Coconut) Oil, Squalane, Tocopherol (Vitamin E), Hyaluronic Acid, Sodium Benzoate, Sodium Levulinate, Potassium Sorbate, and Sodium Phytate. User experience was assessed by subjective participant self-reporting, and responses were collected at times users were able to confidently self-report significant results, ranging between 1-7 days of cream usage. Of this group, 100% reported the cream felt soothing on the skin, significantly reduced discomfort, irritation and itching, visibly reduced redness and inflammation, and left the skin appearing visibly more healthy.

### Example 5

### Rosacea

3 individuals with rosacea, including White ethnicities, ranging from 25-65+ age groups, topically applied a cream composition containing hemocyanin to areas of the body affected by rosacea, including the Face, Back, Chest and Neck areas. The composition contained Water (Aqua), Hemocyanin (Marine Glycoprotein), Butyrospermum Parkii Shea Butter Fruit, Cetyl Alcohol, Glyceryl Stearate, Glycerin, Caprylic/Capric Triglyceride, Sorbitan Oleate Decylglucoside Crosspolymer, Polyacrylamide, C13-14 Isoparaffin, Laureth-7, Cocos Nucifera (Coconut) Oil, Squalane, Tocopherol (Vitamin E), Hyaluronic Acid, Sodium Benzoate, Sodium Levulinate, Potassium Sorbate, and Sodium Phytate. User experience was assessed by subjective participant self-reporting, and responses were collected at times users were able to confidently self-report significant results, ranging between 1-7 days of cream usage. Of this group, 100% reported the cream felt soothing on the skin, significantly reduced discomfort, irritation and itching, visibly reduced redness and inflammation, and left the skin appearing visibly more healthy.

### Example A

### Atopic Dermatitis

Thirty-three female and male subjects, aged 12 to 70 years of age, having Fitzpatrick skin types II-V completed a four-week study using study composition comprising lobster derived glycoprotein applied twice daily and as needed. The composition is the Soothing Hydration Cream obtained from Marin, Brands, Inc., 35 Tee Drive, Portland, Maine, 04103 U.S.A. containing (in weight percent) Water (Aqua) 25-50%, Hemocyanin (Marine Glycoprotein) 20%, Butyrospermum Parkii Shea Butter Fruit 5-15%, Cetyl Alcohol 2-5%, Glyceryl Stearate 2-5%, Glycerin 2-10%, Caprylic/Capric Triglyceride 0.25-5%, Sorbitan Oleate Decylglucoside Crosspolymer 0.25-5%, Polyacrylamide 0.25-5%, C13-14 Isoparaffin, 0.25-5%, Laureth-7 0.25-5%, Cocos Nucifera (Coconut) Oil 0.25-5%, Squalane 0.25-5%, Tocopherol (Vitamin E) 0.05-5%, Hyaluronic Acid 0.05-5%, Sodium Benzoate 0.25-5%, Sodium Levulinate 0.25-5%, and Sodium Phytate 0.05-2%. Clinical evaluations of dryness and skin smoothness (10-point scale), Investigator's Global Assessment (IGA) for Atopic Dermatitis and Eczema Area and Severity Index (EASI), as well as digital imaging of target lesions and subject self-assessment questionnaires, were performed at various timepoints at baseline, immediately post test product application, at 48 hours, at week 1, and week 4 post test product application. Results show that in as early as 48 hours clinical evaluations showed significant improvements (p<0.05) in IGA and EASI scoring, with lasting improvement at week 4. 78.8% of subjects self-reported immediate relief following initial application, and at week 4 90.9% reported skin was less itchy, flaky, red and dry.

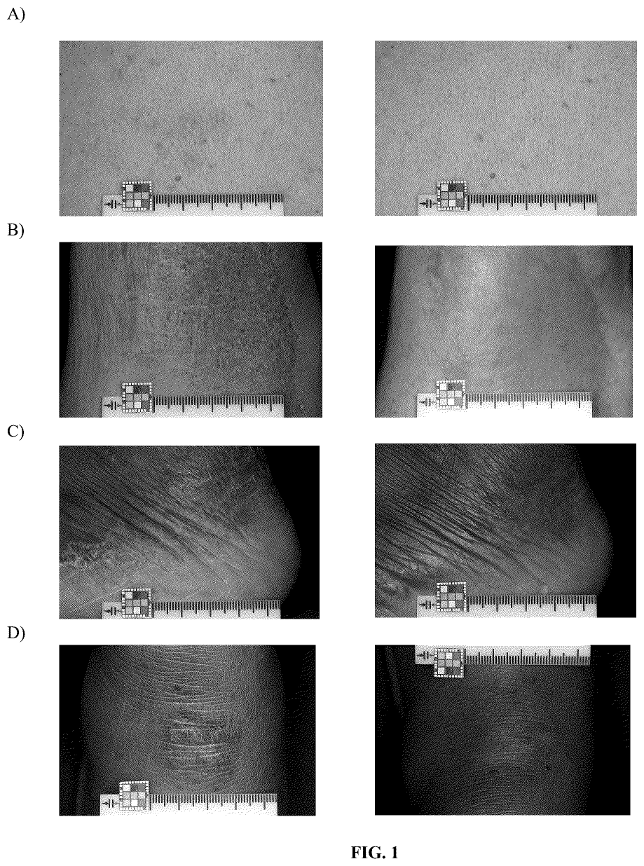

As shown in FIG. 1, digital images of target lesions show comparison of baseline to week 4 post test product application for a variety of skin types A) head and neck, B) trunk, C) upper extremities, and D) lower extremities.

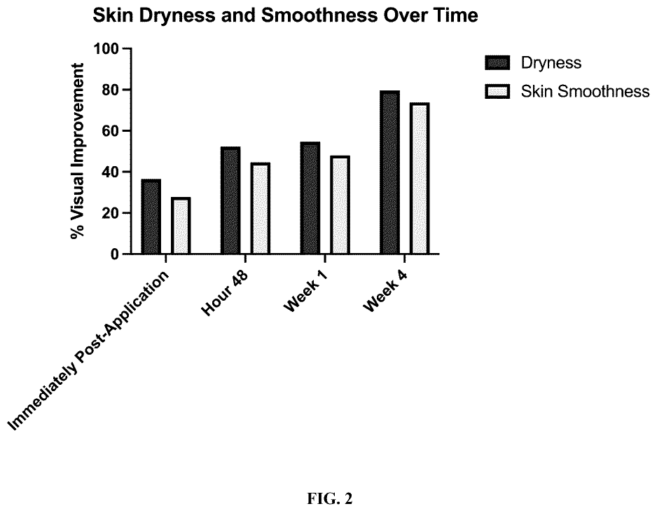

As shown in FIG. 2, a bar graph shows percent improvement of skin dryness and smoothness at various time points post application from immediately post application, 48 hours post application, 1 week post application, and 4 weeks post application.

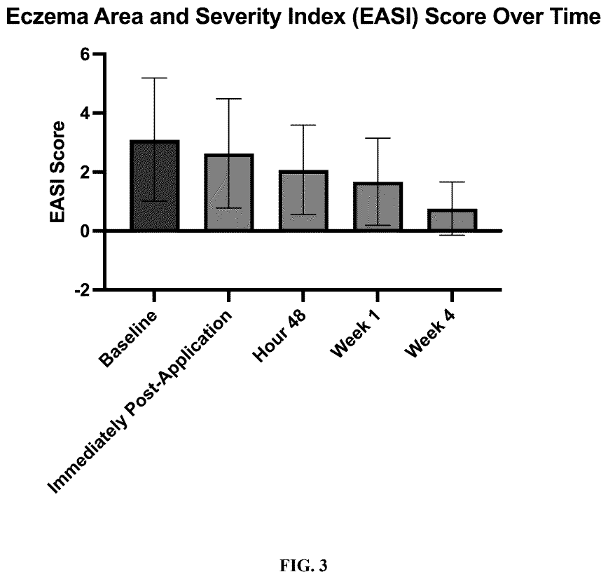

As shown in FIG. 3, a bar graph shows mean EASI score at various time points post application from baseline, immediately post application, 48 hours post application, 1 week post application, and 4 weeks post application (p<0.001 for all time points post application).

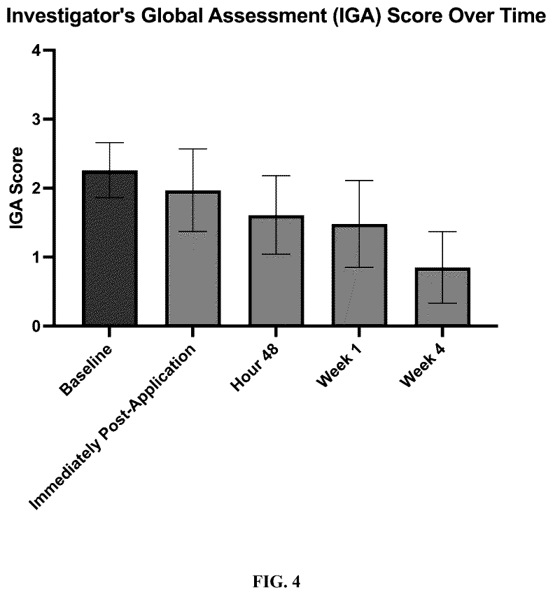

As shown in FIG. 4, a bar graph shows the mean IGA score at various time points post application from baseline, immediately post application, 48 hours post application, 1 week post application, and 4 weeks post application.

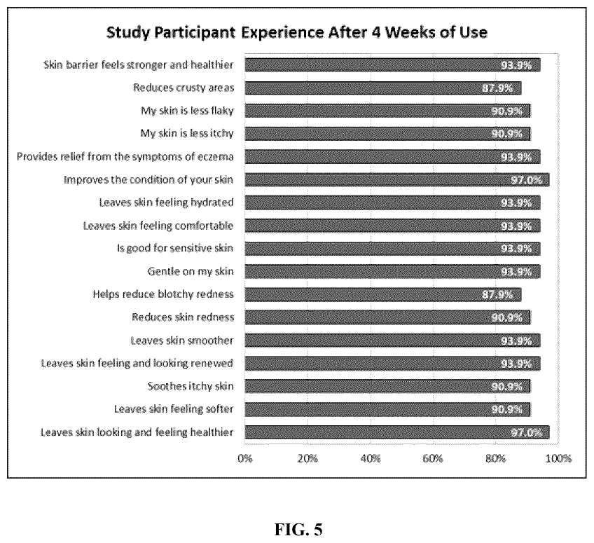

As shown in FIG. 5, a bar graph shows subject agreement level (percent) at study end, displaying a minimum average reported agreement level of 87.9% and most common level of 93.9% over the study 4-week period.

### Results

The test formulation comprising lobster-derived glycoprotein as described above was well accepted by the study subjects. Notably, as shown in FIG. 5, 93.9% reported that the test product provided relief for eczema symptoms, and 90.0% reported that the test formulation soothed itchy skin and reduced erythema, both symptoms associated with atopic dermatitis. These data show that the lobster hemocyanin glycoprotein-based test composition cream significantly reduced the signs of atopic dermatitis, as assessed by clinical evaluations for dryness and smoothness, EASI and IGA scoring, digital imaging, and subject assessment questionnaires, revealing promise for a potentially novel treatment option for atopic dermatitis patients.

### Example B

### Human Clinical Study on Atopic Dermatitis Treatment

The objective of this study was to perform a single-center, monadic clinical study to evaluate the efficacy of a topical cream, formulated with a lobster-derived hemocyanin glycoprotein, in mitigating the signs of atopic dermatitis. To this end, 33 male and female subjects aged 12 to 70, participated in this 4-week Atopic Dermatitis (Eczema) clinical study conducted to evaluate atopic dermatitis treatment with test compositions. A dermatologist made clinical evaluations a time point 0, 48 hours, 1 week, and 4 weeks of dryness and skin smoothness (10-point scale), graded the state of the skin condition via Eczema Area and Severity Index (EASI) and Investigator's Global Assessment (IGA), conducted digital imaging of target lesions at various time points, immediately post test product application, 48 hours, weeks 1, and weeks 4, and subjects completed self-assessment questionnaires at each check-in.

### Test Formulation

The test formulation comprises lobster-derived hemocyanin (marine glycoprotein) active ingredient at a concentration of 10-20% by weight, corresponding to 6-10 mg/mL, or a mass dosage of 3 mg to 30 mg (assuming test subjects applied 0.5 mL to 3 mL of product per use). The test formulation further comprised the following excipients to form a topical cream containing (in weight percent) Water (Aqua) 25-50%, Hemocyanin (Marine Glycoprotein) 20%, Butyrospermum Parkii Shea Butter Fruit 5-15%, Cetyl Alcohol 2-5%, Glyceryl Stearate 2-5%, Glycerin 2-10%, Caprylic/Capric Triglyceride 0.25-5%, Sorbitan Oleate Decylglucoside Crosspolymer 0.25-5%, Polyacrylamide 0.25-5%, C13-14 Isoparaffin, 0.25-5%, Laureth-7 0.25-5%, Cocos Nucifera (Coconut) Oil 0.25-5%, Squalane 0.25-5%, Tocopherol (Vitamin E) 0.05-5%, Hyaluronic Acid 0.05-5%, Sodium Benzoate 0.25-5%, Sodium Levulinate 0.25-5%, and Sodium Phytate 0.05-2%. The test composition (Marin Soothing Hydration Cream) was obtained from Marin Brands, Inc., 35 Tee Drive, Portland, Maine, 04103 U.S.A.

### Inclusion Criteria

The inclusion criteria for the atopic dermatitis clinical study are as follows: (1) Female or male, 12 to 70 years of age. (2) In good general health (physical, mental, and social well-being, not merely the absence of disease/infirmity), according to subject self-report. (3) Having Fitzpatrick skin type I-VI (refer to Appendix I: Fitzpatrick Skin Type). (4) Having mild to moderate active atopic dermatitis on multiple areas of the body at baseline as determined by a board-certified dermatologist-trained Investigator or designee using Rajka and Langeland scale (score 3-7.5 to qualify). Refer to Appendix II: Rating According to Rajka and Langeland Severity Index. (5) Qualifies with atopic dermatitis according to the Atopy Questionnaire. Refer to Appendix III: Atopy Questionnaire for Subject Qualification. (6) If currently on a stabilized regimen of topical steroids or prescription topical eczema medication (e.g. ELIDEL, CUTIVATE, hydrocortisone, MIMYX, etc.), willing to bring to the clinic for documentation at baseline and to continue using for the duration of the study. (7) Willing to continue using regular, nonmedicated body cleansing products. (8) Willing to stop using current body moisturizers (if used) and use only the test product for moisturizing for the duration of the study period. (9) Males whose affected area is the face/neck must be without facial hair (e.g., no beard, mutton chops, more than 24-48 hours growth, etc.) so the face and neck are visible for assessments, and those who are able to shave regularly must be willing to shave within 24-48 hours of each visit (a small mustache and/or goatee is allowed as long as the majority of the face and neck are shaved and the affected area is visible). (10) Who normally wash or bathe daily. (11) Willing to have the skin assessed on the head/neck, trunk (includes internal axillae, but not groin), upper extremities (includes external axillae and hands), and lower extremities (includes feet, but not buttocks). (12) Willing, or having a parent/legal guardian (for minor subjects) who is willing, to provide written informed consent and able to read, speak, write, and understand English. (13) If age 12 to 17, willing to read, understand, and sign the assent form. (14) Willing, or having a parent/legal guardian who is willing (for minor subjects), to sign a photography release. (15) For those with the affected area on the face, having not had any facial treatments in the past 6 months and are willing to withhold all facial treatments during the course of the study including facials, facial peels, photo facials, laser treatments, dermabrasion, botulinum toxin (BOTOX®), injectable filler treatments, intense pulsed light (IPL), acid treatments, tightening treatments, facial plastic surgery, or any other treatment administered by a physician or skin care professional designed to improve the appearance or firmness of facial skin. Waxing and threading are allowed but not facial laser hair removal. (16) Willing to, and/or having a parent/guardian who is willing to (for minor subjects), cooperate and participate by following study requirements (including those outlined in section 7.3) for the duration of the study and to report any changes in health status or medications, AE symptoms, or reactions immediately. (17) If under age 18, having a parent/guardian who is 18 years of age or older who presents proof of guardianship (e.g., birth certificate, adoption certificate; insurance card is acceptable only if both parent/legal guardian and child's name are present on the card) at the baseline visit.

### Exclusion Criteria

The exclusion criteria for the atopic dermatitis clinical study are as follows: (1) Having been diagnosed with known allergies to skin care products. (2) Having a known reactivity to coconut or Hemocyanin (Marine Glycoprotein). (3) Breastfeeding, pregnant, or planning to become pregnant during the study according to subject self-report. (4) Having a pregnant partner or a partner who is planning to become pregnant during the study period or is unwilling or unable to use an acceptable method of contraception. (5) Having a history of skin cancer within the past 5 years. (6) Having clinically active bacterial, fungal, or viral skin infections or those who have a history of skin infections, according to subject self-report. (7) Having a health condition and/or pre-existing or dormant dermatologic disease on the test area (e.g., psoriasis, rosacea, moderate to severe acne, acne conglobata, nodules, or cysts), seborrheic dermatitis, severe excoriations) that the Investigator or designee deems inappropriate for participation or could interfere with the outcome of the study. (8) Having observable sunburn, suntan, scars, nevi, excessive hair, tattoos, or other dermal conditions on the test areas that might influence the test results in the opinion of the Investigator or designee. (9) Having a history of immunosuppression/immune deficiency disorders (including HIV infection, AIDS, multiple sclerosis, Crohn's disease, rheumatoid arthritis), organ transplant (heart, kidney, etc.), or currently using oral or systemic immunosuppressive medications and biologics (e.g., azathioprine, belimumab, CIMZIA®, COSENTYX®, cyclophosphamide, cyclosporine, ENBREL®, HUMIRA®, IMURAN®, KINERET®, mycophenolate mofetil, methotrexate, ORENCIA®, prednisone, REMICADE®, RITUXAN®, SILIQ™, SIMPONI®, STELARA®, TALTZ®) and/or undergoing radiation or chemotherapy as determined by study documentation. (10) Currently using or having regularly used corticosteroids (systemic or topical, not nasal or ocular) within the past 4 weeks (including but not limited to betamethasone, clobetasol, desoximetasone, diflorasone, fluocinonide, halcinonide, and halobetasol). (11) Having a disease such as asthma, diabetes, epilepsy, hypertension, hyperthyroidism, or hypothyroidism that is not controlled by diet or medication. Individuals having multiple health conditions may be excluded from participation even if the conditions are controlled by diet, medication, etc. (12) Having started a long-term medication within the last 2 months. (13) Having any planned surgeries or invasive medical procedures during the study. Noninvasive medical procedures or surgeries will be reviewed for their impact on the study outcome and acceptability by the Investigator or designee. (14) Currently participating in any other clinical trial at SGS or another research facility or doctor's office. (15) Having participated in any clinical trial involving the test area within 2 weeks before study enrollment at SGS or another research facility or doctor's office. (16) Having started hormone replacement therapies (HRT) or hormones for birth control or menopause less than 3 months before study enrollment or who plan on starting, stopping, or changing doses of HRT or hormones for birth control during the study. (17) Having started prescription testosterone therapy less than 3 months prior to study entry or planning on starting, stopping, or changing doses of testosterone therapy during the study (e.g., testosterone cypionate, testosterone enanthate, testosterone pellet, testosterone undecanoate) or on a testosterone booster or prescription testosterone (e.g., DHEA, OMNADREN®, SUSTANON®, testosterone cypionate, testosterone enanthate, testosterone propionate, testosterone phenylpropionate, tribulus).

### Study Procedures

This study was a monadic clinical study. Each study subject was given a body moisturizer to apply to their arms, hands, legs and torso twice a day, or more often if needed. Subjects used the test product for 4-weeks. Safety and tolerance were measured by the frequency and incidence of both cutaneous irritation parameters and adverse events. Product benefit was measured by clinical observations and self-assessments. Clinical evaluations were performed as outlined below at baseline, immediately post-test product application, 48 hours, weeks 1, and 4.

### Investigator Grading of Efficacy

The following parameters were evaluated using the indicated scale anchors shown in Table 1:

TABLE 1

| Parameter | 0= | 9= |
|---|---|---|
| Dryness | No dryness | Severe scaling/fissuring |
| (visual) | | |
| Skin | Smooth, even-looking | Rough, uneven-looking skin |
| smoothness | texture, no roughness | texture |
| (visual) | | |

### Eczema Area and Severity Index (EASI) Scoring

EASI was graded and calculated per a Symptom Severity Score rating the 4 clinical manifestations of AD on each body region (described in the text below), with half-point scores used as necessary to accurately describe the clinical condition, as shown in Table 2:

TABLE 2

| Clinical | | | | |
|---|---|---|---|---|
| Manifestation | Absent = 0 | Mild = 1 | Moderate = 2 | Severe = 3 |
| Erythema | None | Faint erythema | Prominent | Deep, intense red |
| | | | redness | color |
| Edema, | None | Dermal swelling | Definite dermal | Dermal swelling, |
| induration, | | in limited areas | swelling of the | indurated skin in |
| papulation | | discernable by | skin in several | widespread areas |
| | | touch | areas | |
| Excoriations | None | Slight evidence of | Linear marks on | Many weepy or |
| | | scratching, no | skin, epidermal | hemorrhagic |
| | | broken skin | (fluid, crusts) or | lesions |
| | | | dermal (blood) | |
| | | | injury | |
| Lichenification | None | Skin markings | Skin markings | Skin markings |
| | | minimally | exaggerated to a | visibly |
| | | exaggerated | crisscross | exaggerated to a |
| | | | pattern | deep crisscross |
| | | | | pattern |

Body Regions and Weighting: The assessment of AD symptom severity was performed on the following 4 body regions: the head and neck (H), the upper extremities (U), the trunk (T), and the lower extremities (L). The buttocks and feet are counted as part of the lower extremities. The internal axillae and groin are counted as part of the trunk. The external axillae and hands are counted in the upper extremities.

Area scores: An area percentage affected by AD was estimated for each of the 4 body regions. The percentage was assigned a proportional score of 0 to 6 during the analysis, according to the following definitions, as shown in Table 3:

TABLE 3

| Area Score | Percentage of Area Affected by Eczema |
|---|---|
| 0 | 0% |
| | (no eczema in this region) |
| 1 | 1%-9% |
| 2 | 10%-29% |
| 3 | 30%-49% |
| 4 | 50%-69% |
| 5 | 70%-89% |
| 6 | 90%-100% |
| | (entire region is affected by eczema) |

Region Score Calculation: The score for each body region will be obtained by multiplying the sum of the symptom severity scores of the 4 clinical manifestations by the area scores (assigned for each body region as shown above). Each body region result will then be multiplied by the constant weighed value assigned to that body region as shown in Table 4 below: Table 4—EASI Body Region Score Calculation for Subjects 8 Years of Age and Older.

TABLE 4

| Body Region | EASI Score |
|---|---|
| Head/Neck | (Erythema + Induration/Edema/Papulation + |
| (H) | Excoriations + Lichenification) × Area × 0.10 |
| Upper | (Erythema + Induration/Edema/Papulation + |
| extremities | Excoriations + Lichenification) × Area × 0.20 |
| (U) | |
| Trunk | (Erythema + Induration/Edema/Papulation + |
| (T) | Excoriations + Lichenification) × Area × 0.30 |
| Lower | (Erythema + Induration/Edema/Papulation + |
| extremities | Excoriations+Lichenification) × Area × 0.40 |
| (L) | |

The final EASI score was obtained by adding the body region scores per the formula as follows:

$$Final EASI Score = ( H ) + ( U ) + ( T ) + ( L )$$

Additionally, the percent of area affected to the nearest 5% (a total body percentage) was documented for each subject.

### Investigator's Global Assessment (IGA)

Investigator's Global Assessment for atopic dermatitis was performed at baseline, post-application, hour 48, and weeks 1 and 4. The efficacy parameters were assessed on the affected area that best represents the condition, as determined by the Investigator or designee, for each subject using the scale below as shown in Table 5:

TABLE 5

| Score | Description |
|---|---|
| 0 = Clear | No inflammatory signs of atopic dermatitis (no erythema, |
| | no induration/papulation, no lichenification, no oozing/ |
| | crusting). Post inflammatory hyperpigmentation and/or |
| | hypopigmentation may be present. |
| 1 = Almost | Barely perceptible erythema, barely perceptible |
| | induration/papulation, clear |
| 2 = Mild | Slight but definite erythema (pink), slight but definite |
| | induration/papulation, and/or slight but definite |
| | lichenification. No oozing or crusting. |
| 3 = Moderate. | Clearly perceptible erythema (dull red), clearly |
| | perceptible induration/papulation, and/or clearly |
| | perceptible lichenification. Oozing and crusting may |
| | be present |
| 4 = Severe | Marked erythema (deep or bright red), marked |
| | induration/papulation, and/or marked lichenification. |
| | Disease is widespread in extent. Oozing or crusting may |
| | be present. |

Digital Imaging Using TWINFLASH: Digital imaging using TWINFLASH was performed at baseline and weeks 1 and 4. Subjects assessed their tolerability and skin condition per the scales as shown below at baseline, immediately post-test product application, 48 hours, week 1 and week 4 visits. In addition, immediately post-test product application, at 48 hours, weeks 1, and 4 subjects completed a self-assessment questionnaire. Table 6 below shows the qualitative score scales for skin conditions assessed.

TABLE 6

| | Skin | Skin | Skin | Skin | Skin | Skin flakiness and |
|---|---|---|---|---|---|---|
| Scale Score | Texture | Smoothness | softness | moisturization | Dryness | scaling |
| 0 = None | Very Smooth | Very | Very soft | Feels very | Not dry | No apparent flaking |
| | | smooth | | moisturized | at all | or scaling |
| 1 = Mild | | | | | | |
| 2 = Moderate | | | | | | |
| 3 = Severe | Very Rough | Not smooth | Not soft | Not feeling | Very dry | Very apparent flaking |
| | | | | moisturized | | or scaling |

| | Overall skin | Overall skin | Itchiness | | Overall look |
|---|---|---|---|---|---|
| Scale Score | irritation | discomfort | of skin | Skin redness | and feel of skin |
| 0 = None | No apparent | No apparent | Not at all | No apparent | Excellent |
| | irritation | discomfort | itchy | redness | |
| 1 = Mild | | | | | |
| 2 = Moderate | | | | | |
| 3 = Severe | Very irritated | Very | Very itchy | Very red | Poor |
| | | uncomfortable | | | |

### Results—Data

Clinical Improvement: The percentage improvement in dryness and skin smoothness and the reduction percentage in EASI and IGA are shown in Table 7 below: Table 7—Percentage improvement in dryness and skin smoothness and the reduction percentage in EASI and IGA. Improvements statistically significant at p<0.05.

TABLE 7

| | Immediate Post | | | |
|---|---|---|---|---|
| | Application | 48 hours | Week 1 | Week 4 |
| | (Incidence of | (Incidence of | (Incidence of | (Incidence of |
| | improvement %) | improvement %) | improvement %) | improvement %) |
| Dryness | 36.5% (100%) | 52.3% (100%) | 54.7% (97.0%) | 79.6% (100%) |
| (Affected area) | | | | |
| Skin Smoothness | 27.7% (84.8%) | 44.6% (100%) | 48.0% (97.0%) | 73.8% (100%) |
| (Affected area) | | | | |
| IGA | −12.8% (36.4%) | −28.9% (66.7%) | −34.2% (78.8%) | −62.4% (100%) |
| EASI | −15.1% (75.8%) | −33.3% (93.9%) | −46.2% (90.9%) | −75.4% (100%) |
| Affected Area | 0% | −5.7% | −17.2% | −45.3% |

Table 8—Improvement (%) in Self-Assessed Tolerability of the Affected Area—improvements statistically significant at p<0.05.

TABLE 8

| | Immediate Post | | | |
|---|---|---|---|---|
| | Application | 48 hours | Week 1 | Week 4 |
| | (Incidence of | (Incidence of | (Incidence of | (Incidence of |
| | improvement %) | improvement %) | improvement %) | improvement %) |
| Texture | 24.7% (45.5%) | 30.1% (54.5%) | 47.9% (72.7&) | 58.9% (84.8%) |
| Smoothness | 33.3% (57.6%) | 29.2% (51.5%) | 45.8% (63.6%) | 56.9% (81.8%) |
| Softness | 27.5% (48.5%) | 31.9% (57.6%) | 40.6% (66.7%) | 63.8% (78.8%) |
| Moisturization | 46.3% (72.7%) | 50.0% (72.7%) | 56.1% (78.8%) | 78.0% (97.0%) |
| Dryness | 42.9% (69.7%) | 42.9% (69.7%) | 48.1% (66.7%) | 66.2% (87.9%) |
| Flakiness and | 44.1% (57.6%) | 48.5% (57.6%) | 52.9% (60.6%) | 73.5% (84.8%) |
| scaling | | | | |
| Overall irritation | 41.0% (60.6%) | 52.7% (75.8%) | 58.1% (75.8%) | 74.3% (90.9%) |
| Overall skin | 39.1% (60.6%) | 59.4% (75.8%) | 55.1% (72.7%) | 76.8% (90.9%) |
| discomfort | | | | |
| Itchiness | 46.6% (66.7%) | 54.8% (72.7%) | 58.9% (72.7% | 72.6% (84.7%) |
| Redness | 28.6% (48.5%) | 37.5% (51.5%) | 42.9% (54.5%) | 60.7% (66.7%) |
| Overall look and | 34.2% (60.6%) | 38.2%(66.7%) | 50.0% (75.8%) | 69.7% (87.9%) |
| feel of skin | | | | |

TABLE 9
Table 9 - Subject Agreement Percent (%) and Disagreement Percent (%) Results

| | | Immediate | | | | | | | |
|---|---|---|---|---|---|---|---|---|---|
| | | Post | | | | | | | |
| | | Application | | 48 hours | | Week 1 | | Week 4 | |
| | | Agree | Disagree | Agree | Disagree | Agree | Disagree | Agree | Disagree |
| | | Strongly/ | Strongly/ | Strongly/ | Strongly/ | Strongly/ | Strongly/ | Strongly/ | Strongly/ |
| | | Agree | Disagree | Agree | Disagree | Agree | Disagree | Agree | Disagree |
| | | (%) | (%) | (%) | (%) | (%) | (%) | (%) | (%) |
| 1. | Soothes my skin | 72.7% | 0% | 84.8% | 6.1% | 97.0% | 3.0% | 93.9% | 0% |
| 2. | Leaves skin looking | 60.6% | 3.0% | 78.8% | 6.1% | 87.9% | 3.0% | 97.0% | 0% |
| | and feeling | | | | | | | | |
| | healthier | | | | | | | | |
| 3. | Leaves skin feeling | 84.8% | 3.0% | 78.8% | 6.1% | 75.8% | 3.0% | 90.9% | 3.0% |
| | softer | | | | | | | | |
| 4. | Soothes itchy skin | 69.7% | 3.0% | 84.8% | 6.1% | 84.8% | 3.0% | 90.9% | 0% |
| 5. | Leaves skin feeling | 72.7% | 6.1% | 75.8% | 3.0% | 81.8% | 0% | 93.9% | 0% |
| | and looking | | | | | | | | |
| | renewed | | | | | | | | |
| 6. | Leaves skin smoother | 72.7% | 6.1% | 75.8% | 6.1% | 90.9% | 3.0% | 93.9% | 3.0% |
| 7. | Reduces skin redness | 54.5% | 6.1% | 69.7% | 12.1% | 72.7% | 6.1% | 90.9% | 3.0% |
| 8. | Helps reduce | 54.5% | 9.1% | 69.7% | 12.1% | 75.8% | 3.0% | 87.9% | 3.0% |
| | blotchy redness | | | | | | | | |
| 9. | Gentle on my skin | 81.8% | 3.0% | 93.9% | 3.0% | 93.9% | 0% | 93.9 | 3.0% |
| 10. | Is good for | 72.7% | 6.1% | 78.8% | 3.0% | 87.9% | 0% | 93.9% | 3.0% |
| | sensitive skin | | | | | | | | |
| 11. | Leaves skin feeling | 78.8% | 6.1 | 84.8% | 6.1% | 87.9% | 3.0% | 93.9% | 3.0% |
| | comfortable | | | | | | | | |
| 12. | Leaves skin feeling | 84.8% | 3.0% | 84.8% | 3.0% | 87.9% | 6.1% | 93.9% | 3.0% |
| | hydrated | | | | | | | | |
| 13. | Improves the | 72.7% | 0% | 87.9% | 3.0% | 90.9% | 3.0% | 97.0% | 3.0% |
| | condition of your | | | | | | | | |
| | skin | | | | | | | | |
| 14. | Provides relief | 78.8% | 0% | 87.9% | 6.1% | 87.9% | 6.1% | 93.9% | 3.0% |
| | from the symptoms | | | | | | | | |
| | of eczema | | | | | | | | |
| 15. | My skin is less itchy | 75.8% | 3.0% | 84.8% | 3.0% | 84.8% | 3.0% | 90.9% | 3.0% |
| 16. | My skin is less flaky | 81.8% | 0% | 81.8% | 9.1% | 87.9% | 3.0% | 90.9% | 3.0% |
| 17. | Reduces crusty areas | 72.7% | 0% | 72.7% | 6.1% | 84.8% | 3.0% | 87.9% | 3.0% |
| 18. | Skin barrier feels | 72.7% | 0% | 78.8% | 6.1% | 84.8% | 3.0% | 93.9% | 3.0% |
| | stronger and healthier | | | | | | | | |

### Results Summary

The data show that the test formulation featuring a lobster-derived glycoprotein test-formulation was well accepted by the subjects. Notably, 93.9% of subjects self-reported that the test product provided relief for their eczema symptoms and 90.9% agreed that it soothed itchy skin and reduced erythema. In as early as 48 hours clinical evaluations showed significant improvements (p<0.05) in IGA and EASI scoring, with lasting improvement at week 4. 78.8% of subjects self-reported immediate relief following initial application, and at week 4 90.9% reported skin was less itchy, flaky, red and dry. The lobster hemocyanin glycoprotein-based cream significantly reduced the signs of atopic dermatitis, as assessed by clinical evaluations for dryness and smoothness, EASI and IGA scoring, digital imaging, and subject self-assessment questionnaires, revealing promise for a potentially novel treatment option for atopic dermatitis patients.

### Example C

Human user clinical data on additional dermatitis indications: eczema/atopic dermatitis, seborrheic dermatitis, radiation or chemotherapy related dermatitis, perioral dermatitis, psoriasis, rosacea, acne, rashes, redness, itching, topical steroid withdrawal (TSW), and sensitive skin. The test formulation comprises lobster-derived hemocyanin (marine glycoprotein) active ingredient at a concentration of 10-20% by weight, corresponding to 6-10 mg/mL, or a mass dosage of 3 mg to 30 mg (assuming test subjects applied 0.5 mL to 3 mL of product per use). The test composition further containing (in weight percent) Water (Aqua) 25-50%, Hemocyanin (Marine Glycoprotein) 10-20%, Butyrospermum Parkii Shea Butter Fruit 5-15%, Cetyl Alcohol 2-5%, Glyceryl Stearate 2-5%, Glycerin 2-10%, Caprylic/Capric Triglyceride 0.25-5%, Sorbitan Oleate Decylglucoside Crosspolymer 0.25-5%, Polyacrylamide 0.25-5%, C13-14 Isoparaffin, 0.25-5%, Laureth-7 0.25-5%, Cocos Nucifera (Coconut) Oil 0.25-5%, Squalane 0.25-5%, Tocopherol (Vitamin E) 0.05-5%, Hyaluronic Acid 0.05-5%, Sodium Benzoate 0.25-5%, Sodium Levulinate 0.25-5%, and Sodium Phytate 0.05-2% (Marin Soothing Hydration Cream) was obtained from Marin Brands, Inc., 35 Tee Drive, Portland, Maine, 04103 U.S.A.

Eczema/atopic dermatitis: 1,469 individuals with eczema/Atopic Dermatitis], including Asian, African American, Hispanic, and White ethnicities, from toddler to 65+ age groups, topically applied a cream composition containing hemocyanin to areas of the body affected by the concern, including the Face, Neck, Scalp, Nose, Eyelid, Perioral, Armpit, Upper arm, Elbow, Forearm, Hand, Finger Stomach, Chest, Lower and Upper Back, Thigh, Shin, Knee, Feet areas. The composition tested was as described above. User experience was assessed by subjective participant self-reporting, and responses were collected at times users were able to confidently self-report significant results, ranging between 1-30 days of cream usage.

Results: Of this group, 81% reported the cream significantly reduced the symptoms of Eczema/Atopic Dermatitis, including reduced itching, left the skin feeling significantly hydrated and soothed, visibly reduced dryness, flaking, redness and inflammation, visibly reduced the appearance of a flare up, and/or left the skin appearing visibly more healthy.

Seborrheic dermatitis: 9 individuals with Seborrheic Dermatitis, including Asian, African American, Hispanic, and White ethnicities, from toddler to 65+ age groups, topically applied a cream composition containing hemocyanin to areas of the body affected by the concern], including the Face, Neck, Scalp, Nose, Eyelid, Perioral, Armpit, Upper arm, Elbow, Forearm, Hand, Finger Stomach, Chest, Lower and Upper Back, Thigh, Shin, Knee, Feet areas. The composition tested was as described above. User experience was assessed by subjective participant self-reporting, and responses were collected at times users were able to confidently self-report significant results, ranging between 1-30 days of cream usage.

Results: Of this group, 88% reported the cream significantly reduced the symptoms and improved the presentation of seborrheic dermatitis, including reduced itching, reduction in scaling and scabbing, left the skin feeling significantly hydrated and soothed, visibly reduced dryness, flaking, scaling, redness and inflammation, visibly reduced the appearance of a flare up, and/or left the skin appearing visibly more healthy.

Radiation or chemotherapy related dermatitis: 29 individuals with Radiation or Chemotherapy-related Dermatitis, including Asian, African American, Hispanic, and White ethnicities, from toddler to 65+ age groups, topically applied a cream composition containing hemocyanin to areas of the body affected by the concern, including the Face, Neck, Scalp, Nose, Eyelid, Perioral, Armpit, Upper arm, Elbow, Forearm, Hand, Finger Stomach, Chest, Lower and Upper Back, Thigh, Shin, Knee, Feet areas. The composition tested was as described above. User experience was assessed by subjective participant self-reporting, and responses were collected at times users were able to confidently self-report significant results, ranging between 1-30 days of cream usage.

Results: Of this group, 93% reported the cream significantly reduced the symptoms of radiation or chemotherapy-related dermatitis, including reduced itching, left the skin feeling significantly hydrated and soothed, visibly reduced dryness, flaking, redness, rashes and inflammation, visibly reduced the appearance of a flare up, and/or left the skin appearing visibly more healthy.

Perioral dermatitis: 309 individuals with Perioral Dermatitis or Dry, Red, Flaky, Eczema-affected Lips, including Asian, African American, Hispanic, and White ethnicities, from toddler to 65+ age groups, topically applied a cream composition containing hemocyanin to areas of the body affected by such concerns, mainly the Lips and Oral areas. The composition contained either containing (in weight percent) Water (Aqua) 25-50%, Hemocyanin (Marine Glycoprotein) 20%, Butyrospermum Parkii Shea Butter Fruit 5-15%, Cetyl Alcohol 2-5%, Glyceryl Stearate 2-5%, Glycerin 2-10%, Caprylic/Capric Triglyceride 0.25-5%, Sorbitan Oleate Decylglucoside Crosspolymer 0.25-5%, Polyacrylamide 0.25-5%, C13-14 Isoparaffin, 0.25-5%, Laureth-7 0.25-5%, Cocos Nucifera (Coconut) Oil 0.25-5%, Squalane 0.25-5%, Tocopherol (Vitamin E) 0.05-5%, Hyaluronic Acid 0.05-5%, Sodium Benzoate 0.25-5%, Sodium Levulinate 0.25-5%, and Sodium Phytate 0.05-2%, or Formula #2 containing Polyisobutene 50-80%, Caprylic/Capric Triglyceride 5-10%, Squalane 5-10%, Synthetic Beeswax (Non-animal, natural derived) 1-5%, Cocos Nucifera (Coconut) Oil 1-5%, 12-Hydroxystearic Acid 1-5%, Euphorbia Cerifera (Candelilla) Wax 1-5%, Hemocyanin (Marine Glycoprotein) 1%, Glyceryl Adipate 1-5%, Glyceryl Stearate 1-5%, Tocopherol 0.5-3.5%, and Stevia 0.5-3.5% optionally with added flavor, and if flavor is added caprylic/capric triglyceride content is 0.025-3.5%, and Stevia rebaudiana extract 0.025-3.5%. User experience was assessed by subjective participant self-reporting, and responses were collected at times users were able to confidently self-report significant results, ranging between 1-30 days of product usage.

Results: Of this group, 97% reported the cream significantly reduced the symptoms of Perioral Dermatitis or dry, red, flaky, eczema-affected lips, including reduced itching, left the skin feeling significantly hydrated, soft and soothed, visibly reduced dryness, flaking, redness, rashes and inflammation, visibly reduced the appearance of a flare up, and/or left the skin appearing visibly more healthy.

Psoriasis: 224 individuals with Psoriasis, including Asian, African American, Hispanic, and White ethnicities, from toddler to 65+ age groups, topically applied a cream composition containing hemocyanin to areas of the body affected by Psoriasis, including the Face, Neck, Scalp, Nose, Eyelid, Perioral, Armpit, Upper arm, Elbow, Forearm, Hand, Finger Stomach, Chest, Lower and Upper Back, Thigh, Shin, Knee, Feet areas. The composition tested was as described above. User experience was assessed by subjective participant self-reporting, and responses were collected at times users were able to confidently self-report significant results, ranging between 1-30 days of cream usage.

Results: Of this group, 94% reported the cream significantly reduced the symptoms and improved the presentation of Psoriasis, including reduced itching, scaling and scabbing, left the skin feeling significantly hydrated and soothed, visibly reduced dryness, flaking, redness and inflammation, visibly reduced the appearance of a flare up, and/or left the skin appearing visibly more healthy.

Rosacea: 111 individuals with Rosacea, including Asian, African American, Hispanic, and White ethnicities, from toddler to 65+ age groups, topically applied a cream composition containing hemocyanin to areas of the body affected by Rosacea, mainly but not limited to the Face and Neck regions. The composition tested was as described above. User experience was assessed by subjective participant self-reporting, and responses were collected at times users were able to confidently self-report significant results, ranging between 1-30 days of cream usage.

Results: Of this group, 93% reported the cream significantly reduced the symptoms and improved the presentation of Rosacea, including reduced itching, redness, irritation, returning the skin to a normal tone, left the skin feeling significantly hydrated and soothed, visibly reduced dryness and flaking, and/or left the skin appearing visibly more healthy.

Acne: 36 individuals with Acne, including Asian, African American, Hispanic, and White ethnicities, from toddler to 65+ age groups, topically applied a cream composition containing hemocyanin to areas of the body affected by Acne, including the Face, Neck, Scalp, Nose, Eyelid, Perioral, Armpit, Upper arm, Elbow, Forearm, Hand, Finger Stomach, Chest, Lower and Upper Back, Thigh and Shin areas. The composition tested was as described above. User experience was assessed by subjective participant self-reporting, and responses were collected at times users were able to confidently self-report significant results, ranging between 1-30 days of cream usage.

Results: Of this group, 91% reported the cream significantly reduced the symptoms and improved the presentation of acne, including reduced itching, bumpiness, the appearance of acne, clogged pores and pustules, left the skin feeling significantly hydrated and soothed, visibly reduced dryness, flaking, redness and inflammation, and/or left the skin appearing visibly more healthy.

Rashes: 239 individuals with Rashes, including Asian, African American, Hispanic, and White ethnicities, from toddler to 65+ age groups, topically applied a cream composition containing hemocyanin to areas of the body affected by rashes, including the Face, Neck, Scalp, Nose, Eyelid, Perioral, Armpit, Upper arm, Elbow, Forearm, Hand, Finger Stomach, Chest, Lower and Upper Back, Thigh, Shin, Knee, Feet areas. The composition tested was as described above. User experience was assessed by subjective participant self-reporting, and responses were collected at times users were able to confidently self-report significant results, ranging between 1-30 days of cream usage.

Results: Of this group, 86% reported the cream significantly reduced the symptoms and improved the presentation of rashes, including reduced itching, redness, irritation, inflammation and hives, reduced bumpiness and returned the skin to a normal texture, left the skin feeling significantly hydrated and soothed, visibly reduced dryness and flaking, visibly reduced the appearance of a flare up, and/or left the skin appearing visibly more healthy.

Redness: 218 individuals with Redness, including Asian, African American, Hispanic, and White ethnicities, from toddler to 65+ age groups, topically applied a cream composition containing hemocyanin to areas of the body affected by redness, including the Face, Neck, Scalp, Nose, Eyelid, Perioral, Armpit, Upper arm, Elbow, Forearm, Hand, Finger Stomach, Chest, Lower and Upper Back, Thigh, Shin, Knee, Feet areas. The composition tested was as described above. User experience was assessed by subjective participant self-reporting, and responses were collected at times users were able to confidently self-report significant results, ranging between 1-30 days of cream usage.

Results: Of this group, 89% reported the cream significantly reduced the symptoms and improved the presentation of redness, including reduced itching, redness, irritation, returning the skin to a normal tone, left the skin feeling significantly hydrated and soothed, visibly reduced dryness and flaking, and/or left the skin appearing visibly more healthy.

Itching: 810 individuals with Redness, including Asian, African American, Hispanic, and White ethnicities, from toddler to 65+ age groups, topically applied a cream composition containing hemocyanin to areas of itchy, inflamed, irritated areas, including the Face, Neck, Scalp, Nose, Eyelid, Perioral, Armpit, Upper arm, Elbow, Forearm, Hand, Finger Stomach, Chest, Lower and Upper Back, Thigh, Shin, Knee, Feet areas. The composition tested was as described above. User experience was assessed by subjective participant self-reporting, and responses were collected at times users were able to confidently self-report significant results, ranging between 1-30 days of cream usage.

Results: Of this group, 85% reported the cream significantly reduced the symptoms and improved the presentation of seborrheic dermatitis, including reduced itching, reduction in bumpiness and scabbing, left the skin feeling significantly hydrated and soothed, visibly reduced dryness, flaking, redness and inflammation, visibly reduced the appearance of a flare up, and/or left the skin appearing visibly more healthy.

Topical steroid withdrawal (TSW): 31 individuals with Redness, including Asian, African American, Hispanic, and White ethnicities, from toddler to 65+ age groups, topically applied a cream composition containing hemocyanin to areas of the body affected by Topical Steroid Withdrawal, including the Face, Neck, Scalp, Nose, Eyelid, Perioral, Armpit, Upper arm, Elbow, Forearm, Hand, Finger Stomach, Chest, Lower and Upper Back, Thigh, Shin, Knee, Feet areas. The composition tested was as described above. User experience was assessed by subjective participant self-reporting, and responses were collected at times users were able to confidently self-report significant results, ranging between 1-30 days of cream usage.

Results: Of this group, 92% reported the cream significantly reduced the symptoms and improved the presentation of seborrheic dermatitis, including reduced itching, peeling and flaking, reduction in bumpiness and scabbing, left the skin feeling significantly hydrated and soothed, visibly reduced dryness, redness and inflammation, visibly reduced the appearance of a flare up, and/or left the skin appearing visibly more healthy.

Sensitive skin: 410 individuals with Redness, including Asian, African American, Hispanic, and White ethnicities, from toddler to 65+ age groups, topically applied a cream composition containing hemocyanin to areas of sensitive skin, including the Face, Neck, Scalp, Nose, Eyelid, Perioral, Armpit, Upper arm, Elbow, Forearm, Hand, Finger Stomach, Chest, Lower and Upper Back, Thigh, Shin, Knee, Feet areas. The composition tested was as described above. User experience was assessed by subjective participant self-reporting, and responses were collected at times users were able to confidently self-report significant results, ranging between 1-30 days of cream usage.

Results: Of this group, 95% reported the cream significantly reduced the symptoms and improved the presentation of seborrheic dermatitis, including reduced itching, dryness, flaking, redness and inflammation, left the skin feeling significantly hydrated and soothed, visibly reduced the appearance of a flare up, and/or left the skin appearing visibly less thin, more fortified, desensitized and healthy.

The clinical test results are summarized in Table 10 below:

TABLE 10

| | Number of | Result - % |
|---|---|---|
| Indication | subjects tested (N) | improvement |
| Eczema/atopic | 1,469 | 81% |
| dermatitis | | |
| Seborrheic | 9 | 88% |
| dermatitis | | |
| Radiation or | 29 | 93% |
| chemotherapy | | |
| related dermatitis | | |
| Perioral dermatitis | 309 | 97% |
| Psoriasis | 224 | 94% |
| Rosacea | 111 | 93% |
| Acne | 36 | 91% |
| Rashes | 293 | 86% |
| Redness | 218 | 89% |
| Itching | 810 | 85% |
| Topical steroid | 31 | 92% |
| withdrawal | | |
| (TSW) | | |
| Sensitive skin | 410 | 95% |

### Example D

In Vitro study: Fibroblast Viability, Fibroblast Proliferation, Hyaluronic Acid production and Type I Collagen Synthesis Data

A fibroblast cell culture model was used to assess the ability of a lobster hemolymph concentrate mainly, comprising the glycoprotein hemocyanin to exert an effect on Fibroblast Cell Viability and Proliferation, Type I Collagen synthesis and Hyaluronic Acid (HA) production, using a sea water analog buffer (with mineral content, pH and salinity levels matching that of the Casco Bay in Portland, Maine) as the vehicle control. Cell viability and proliferation was measured via MTT Assay, Type I Collagen synthesis using a type I C-peptide ELISA-based method, and Hyaluronic Acid content using an ELISA-based method. The test formulation comprises lobster-derived hemocyanin (marine glycoprotein) active ingredient at various concentrations of 1-20% by weight, corresponding to 0.3-6 mg/mL, or a mass dosage of 0.05 mg to 3 mg.

### Preparation of Fibroblasts

Fibroblasts were seeded into the individual wells of a 24-well plate in 0.5 ml of Fibroblast Growth Media (FGM) and incubated overnight at 37+2oC and 5+1% CO2. On the following day the media was removed via aspiration to eliminate any non-adherent cells and replaced with 0.5 ml of fresh FGM. The cells were grown until confluent, with a media change every 48 to 72 hours. Upon reaching confluency the cells were treated for 24 hours with DMEM supplemented with 1.5% FBS to wash out any effects from the growth factors included in the normal culture media. After this 24-hour wash out period the cells were treated with the test materials at the specified concentrations dissolved in FGM with 1.5% FBS. TGF-B (50 ng/ml) was used as a positive control for collagen and elastin, while 100 μM DbcAMP was used as a positive control for hyaluronic acid. Untreated cells (negative controls) just received DMEM with 1.5% FBS. The cells were incubated for 48 hours and at the end of the incubation period cell culture medium was collected and either stored frozen (−75° C.) or assayed immediately. Materials were tested in triplicate. The mean MTT absorbance value for the negative control cells was calculated and used to represent 100% cell viability. The individual MTT values from the cells undergoing the various treatments were then divided by the mean value for the negative control cells and expressed as a percent to determine the change in cell viability caused by each treatment.

### Type I Collagen Assay

Within the skin's dermal layer, fibroblasts synthesize a large precursor molecule called procollagen, which eventually becomes collagen. During the conversion to mature collagen, a segment known as the type I C-peptide is removed. Both the finished collagen and this C-peptide fragment are secreted into the extracellular space. Because procollagen processing consistently generates one molecule of type I C-peptide for every collagen molecule, measuring the amount of this type I-C peptide or fragment provides an accurate indicator of collagen production. This measurement can be performed using an ELISA (enzyme-linked immunosorbent assay) technique. A series of type I C-peptide standards was prepared ranging from 0 ng/ml to 640 ng/ml. Next, an ELISA microplate was prepared by removing any unneeded strips from the plate frame followed by the addition of 100 μl of peroxidase-labeled anti procollagen type I-C peptide antibody to each well used in the assay. Twenty (20) μl of either sample (collected tissue culture media) or standard was then added to appropriate wells and the microplate was covered and allowed to incubate for 3±0.25 hours at 37° C. After the incubation the wells were aspirated and washed three times with 400 μl of wash buffer. After the last wash was removed 100 μl of peroxidase substrate solution (hydrogen peroxide+tetramethylbenzidine as a chromagen) was added to each well and the plate was incubated for 15±5 minutes at room temperature. After the incubation 100 μl of stop solution (1 N sulfuric acid) was added to each well and the plate was read using a microplate reader at 450 nm.

### Hyaluronic Acid Assay

A series of hyaluronic acid standards was prepared ranging from 50 ng/ml to 3,200 ng/ml. Next, 100 μl of each standard and sample was transferred to a well in an incubation plate. After adding 50 μl of detection solution to each well (except the reagent blank wells) the plate was incubated for 1±0.25 hour at 37±2° C. After the incubation, 100 μl of each sample/standard from the incubation plate was transferred to a corresponding well in the ELISA plate. The ELISA plate was covered and incubated for 30±5 minutes at 4° C. and then washed three times with 300 μl of wash buffer. After the final wash 100 μl of enzyme solution was added to each well and the plate was incubated at 37±2° C. for 30±5 minutes. After this incubation the wells were washed again as described above and then 100 μl of enzyme substrate solution was added to each well and the plate was incubated for 30-45 minutes at room temperature. After this final incubation 50 μl of stop solution was added to each well and the absorbance of the plate was measured at 405 nm using a plate reader. A standard curve created from known concentrations of each substance was used to quantify their amounts, and a regression analysis used to determine the best-fit line for these data points. The absorbance values from test materials and untreated samples were then utilized to estimate the substance quantities in each sample. All treatment means were compared using an ANOVA, with an n=3 per treatment. Statistical significance was set at p<0.05. The symbol (*) used in graphs denotes values that are significantly different from the Untreated group (p<0.05).

### Results

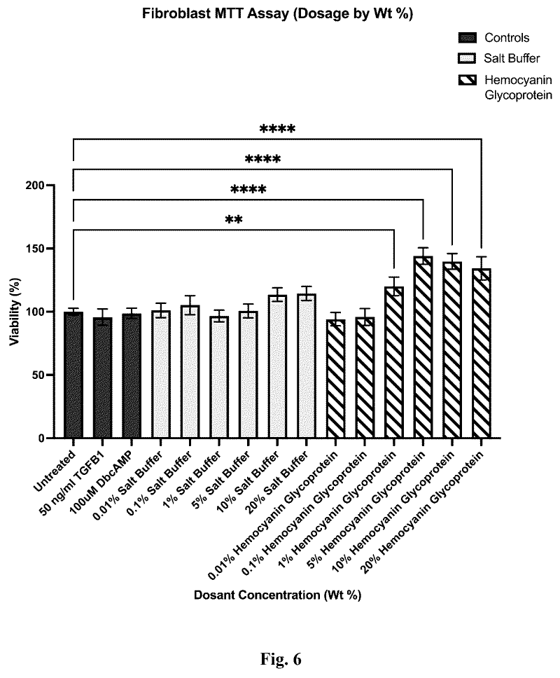

As detailed below, results demonstrated significant increase in fibroblast cell viability, fibroblast cell proliferation, hyaluronic acid content and production at a range of concentrations (dose-dependent response), and an increase in type I collagen synthesis at certain concentrations. As shown in FIG. 6, an MTT assay of fibroblasts of dosage via weight percent shows that relative to the control (untreated) cells measured at 100% viability, 20%, 10%, and 5% Hemocyanin Glycoprotein dosage levels increased fibroblast viability to 134%, 140% and 144%, respectively, indicating an increase in total fibroblast cells—a significant demonstration of fibroblast proliferation occurring across a wide range of the upper dosages.

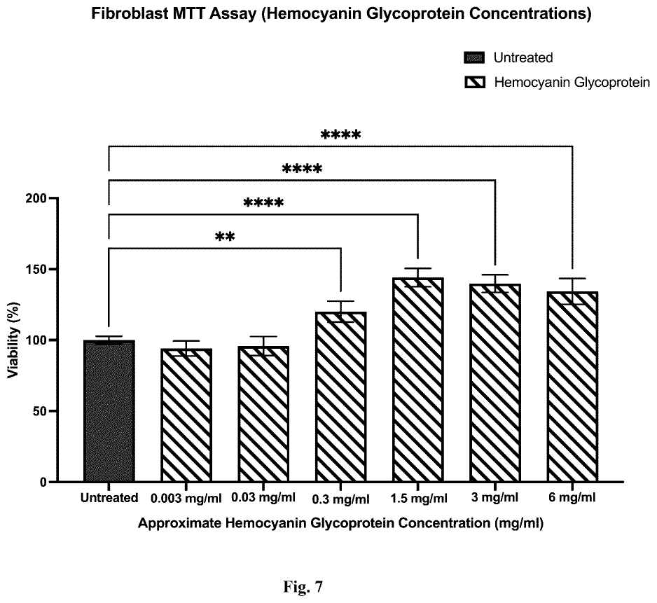

As shown in FIG. 7, an MTT assay data shows data transposed from weight percentage-based dosage, to mg/mL dose concentrations specifically of lobster glycoproteins (Hemocyanin). Relative to the control (untreated) cells measured at 100% viability, 6 mg/mL, 3 mg/mL and 1.5 mg/mL Hemocyanin Glycoprotein dosage levels demonstrated statistically significant increases in fibroblast viability to 134%, 140% and 144%, respectively. Again, this demonstrates an increase in fibroblast cell count, thereby an increase in fibroblast cell proliferation.

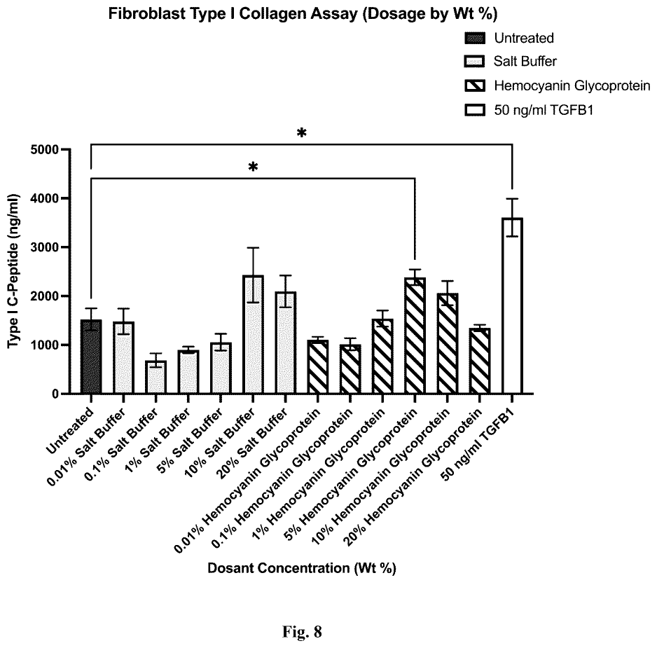

As shown in FIG. 8, a Type I collagen assay shows statistically significant collagen production dosed with the Lobster hemocyanin glycoprotein at 5%.

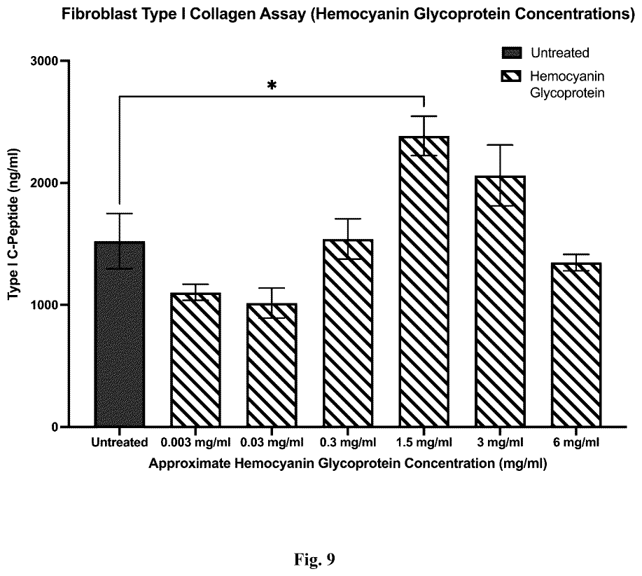

As shown in FIG. 9, a Fibroblast Type I Collagen Assay shows Type I C-Peptide (ng/ml) production with various Hemocyanin Glycoprotein concentrations of 0.003 mg/ml, 0.03 mg/ml, 0.3 mg/ml, 1.5 mg/ml, 3 mg/ml, and 6 mg/ml, with the strongest production at 1.5 mg/ml.

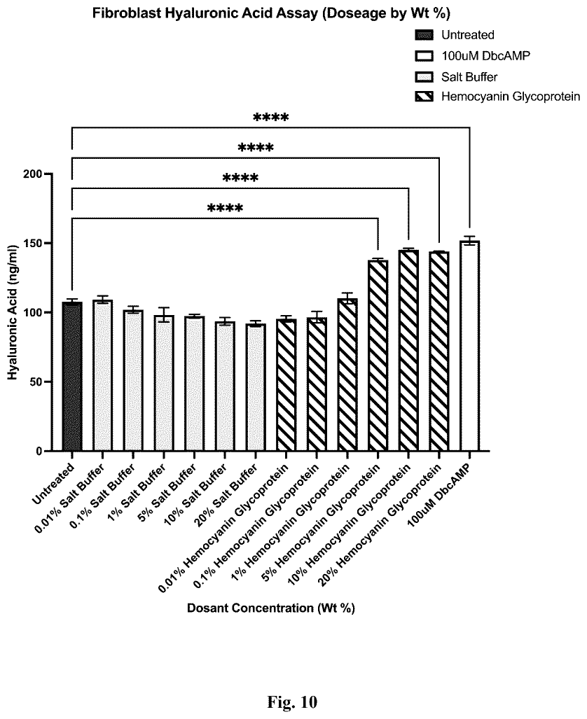

As shown in FIG. 10, a Hyaluronic Acid Assay of fibroblast HA content in ng/ml present in various test composition components shows a statistically significant increase in HA content most notably at 5%, 10%, and 20% Hemocyanin Glycoprotein solution weight percentages, revealing a clear dose-response curve in HA production dosed with hemocyanin glycoproteins.

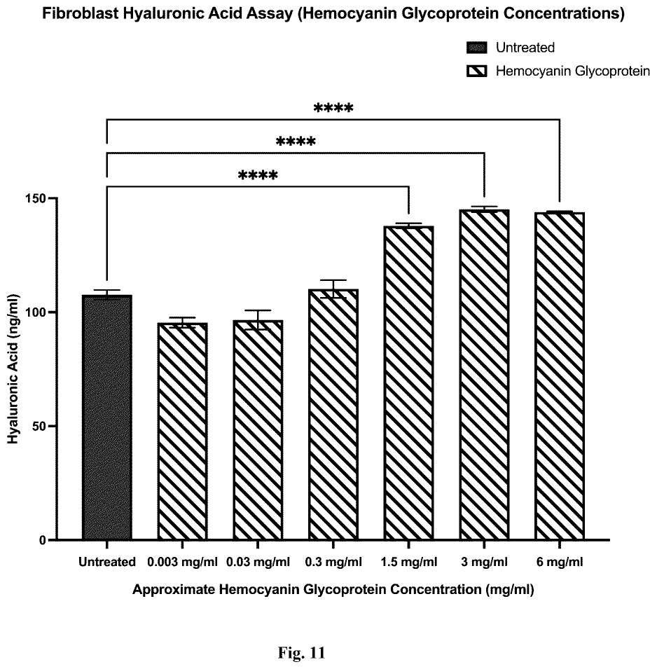

As shown in FIG. 11, results of a Hyaluronic Acid Assay in fibroblasts versus various hemocyanin concentrations show data transposed from weight percentage-based dosage, to mg/mL dose concentrations of Hemocyanin Glycoproteins. Results from this assay demonstrate a statistically significant increase in HA content most notably at 1.5 mg/mL, 3 mg/mL, and 6 mg/mL of hemocyanin glycoproteins, revealing a clear dose-response curve in HA production dosed with hemocyanin glycoproteins.

### Example E

### In Vitro: Anti-Inflammatory Effect on Fibroblasts

This study investigated the viable dosage levels and anti-inflammatory properties of two forms of the lobster glycoprotein hemocyanin, on adult Human Dermal Fibroblasts (HDFa). Approximate hemocyanin glycoprotein dosages followed a range of: 0.01 mg/mL, 0.1 mg/mL, 1 mg/mL, 10 mg/mL, 16 mg/mL and 25 mg/mL, 0.1 mg/mL to 25 mg/mL.

### Hemocyanin Glycoprotein Preparation

Two forms of hemocyanin glycoproteins test formulations were prepared, Hemocyanin Glycoprotein Solution and Clarified Hemocyanin Glycoprotein Solution, as follows: Hemocyanin Glycoprotein Solution was prepared by removing hemocytes and other debris from native lobster hemolymph. Hemocyanin glycoproteins remained in the resulting solution at physiologically normal concentrations. Approximate hemocyanin concentrations were measured and subsequent dilutions to achieve desired test concentrations were performed in a simple salt buffer to mimic ionic concentration of the hemolymph. Clarified Hemocyanin Glycoprotein Solution was prepared by further concentrating the hemocyanin proteins present in the Hemocyanin Glycoprotein Solution as previously described by placing the Hemocyanin Glycoprotein Solution into 10 kDa molecular weight cutoff protein concentrators and centrifuging until about half of the original volume remained in the top portion. The top portion of the solution was collected and approximate hemocyanin concentrations were measured. Subsequent dilutions to achieve desired test concentrations were performed by diluting the top portion in the <10 kDa flowthrough as the dilutant.

### Cell Preparation and Cell Viability

Standard preparation of HDFa cells was conducted. Adult human dermal fibroblasts (HDFa, 5×10⁴ cells per well) were seeded in 48-well plates with complete cell culture media and cultured in humidified incubator at 37° C., 5% CO₂ until cells were confluent. Cells were washed once with PBS and pre-treated for 4 hours with the different test concentrations of raw and concentrated glycoprotein, as well as the positive control (1 μM Dexamethasone). HDFa cells were stimulated using 1 μg/mL lipopolysaccharide (LPS) for 24 hours. Post incubation, supernatants were collected and stored at −80° C. for IL-la and IL-6 ELISA analysis. The kits used for the assay were human IL-6 ELISA. ELISAs were performed in accordance with manufacturer's instructions. To summarize, the Capture Antibody was added to the wells of a clear 96 well plate and incubated overnight at room temperature. The next day, the plate was washed twice with 400 μL Wash Buffer and blocked with Reagent Diluent for 1 ℏr at room temperature. The wells were washed twice with 400 μL Wash Buffer again before standards and diluted samples were added and incubated for 2 hours at room temperature. The plate was washed twice more with 400 μL Wash Buffer before Detection Antibody was added and incubated for 2 hrs at room temperature. The plate was washed again twice followed by the addition of Streptavidin-HRP and incubated at room temperature for 20 mins. The plate was washed a final two times before Substrate Solution was added for 20 mins at room temperature. Stop Solution was added and absorbance was read at 450 nm and 540 nm (or 570 nm to correct for plate optical imperfections) using a Varioskan Lux Plate reader (Thermo Scientific).

A cytotoxicity assay was run to determine appropriate dosage ranges for HDFa cells to maintain viability. Cells were dosed with resazurin reagent following kit instructions (Abcam Resazurin Assay Kit ab129732, abcam.com) and fluorescence was measured at 560 nm/590 nm using a plate reader. The resazurin assay is a fluorometric/colorimetric assay used to measure the metabolic capacity of live cells. It does so by measuring the reduction of oxidized non-fluorescent blue resazurin to a red fluorescent dye (resorufin) by the mitochondrial respiratory chain in live cells. The amount of resorufin produced is directly proportional to the number of living cells. Resorufin has Ex/Em of 530-560/590 nm and absorbance at 570 nm.

### Anti-Inflammatory Assay

Appropriate controls and Hemocyanin Glycoproteins in a range of dosages were added to HDFa cells for four hours prior to the addition of the inflammatory stimuli, lipopolysaccharides (LPS) at a concentration of 1 μg/ml. After the addition of LPS the culture was incubated for 24 hrs and an ELISA-based assay was performed to measure the level of the pro-inflammatory cytokine IL-6. Approximate Hemocyanin Glycoprotein dosages followed a range of: 0.01 mg/ml, 0.1 mg/ml, 1 mg/ml, 10 mg/ml, 16 mg/ml and 25 mg/ml.

### Results

The inflammation assay demonstrated a significant reduction in levels of the pro-inflammatory cytokine, IL-6 in HDFa after treatment with Hemocyanin Glycoproteins at approximate concentrations between 10 mg/ml and 25 mg/ml. The reduction was even greater than that observed with the corticosteroid positive control tested, dexamethasone (1 μM), indicating strong anti-inflammatory effects of the Hemocyanin Glycoprotein.

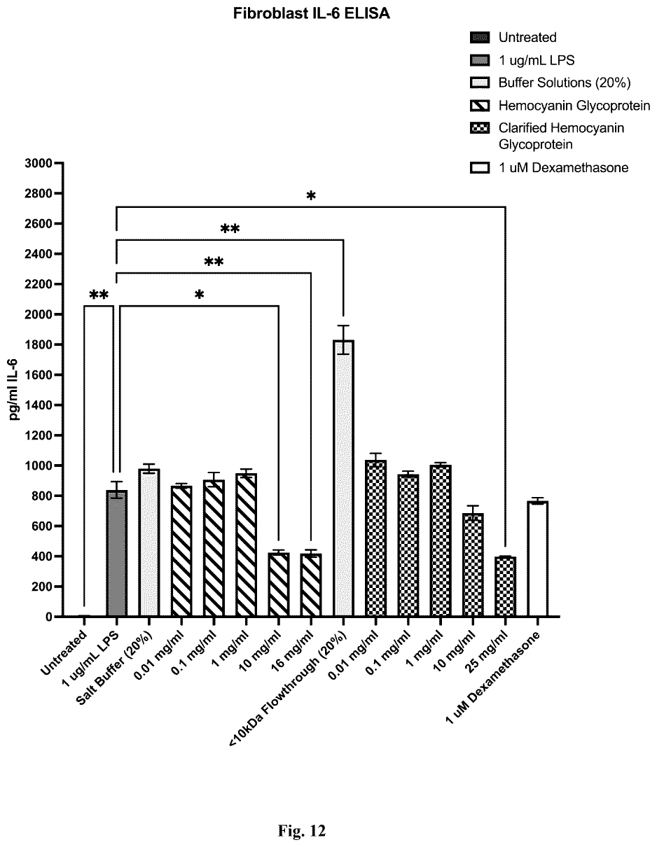

As shown in FIG. 12, treatment with 1 μg/ml of LPS alone caused a significant increase in the level of IL-6 compared to the cells left untreated (**p adj.<0.01). Treatment with raw Glycoprotein Solution at both 10 mg/ml and 16 mg/ml, and with Clarified Hemocyanin Solution at 25 mg/ml resulted in significantly decreased IL-6 levels compared to the LPS group alone (*p adj.<0.05, **p adj.<0.01). Treatment with 1 μM of the corticosteroid, dexamethasone resulted in a decrease in the mean level of IL-6 compared to LPS alone, however, statistically significant reduction was not observed (n.s., p=0.56). It is unclear why 10 mg/ml of the raw Glycoprotein Solution significantly decreased the levels of IL-6, but 10 mg/ml of Clarified Hemocyanin Solution did not, however, the mean was lower than the LPS group alone, but not in a statistically significant manner (10 mg/ml Concentrated Hemocyanin Solution mean=686 pg/ml vs 1 μg/ml LPS mean=837 pg/ml, p adj.=0.15). Overall, this data provides evidence of a strong anti-inflammatory effect of Hemocyanin Glycoproteins.

### Example E1

### In Vitro Hemocyanin Glycoprotein Antioxidant Dermal Fibroblast and Keratinocyte Study

This study investigated the viable dosage levels and antioxidant properties of the Lobster Glycoprotein, Hemocyanin, on adult Human Dermal Fibroblasts (HDFa) and Human adult low Calcium high Temperature (HaCaT) Keratinocytes. Approximate Glycoprotein dosages in fibroblasts followed a range of: 0.01 mg/ml, 0.1 mg/mL, 1 mg/mL, 10 mg/mL, 16 mg/mL and 25 mg/mL. Approximate Hemocyanin Glycoprotein dosages in keratinocytes followed a range of: 0.01 mg/ml, 0.1 mg/mL, 1 mg/mL, 2 mg/mL, and 4 mg/mL.

### Test Methods

Two forms of hemocyanin glycoproteins test formulations were prepared, Hemocyanin Glycoprotein Solution and Clarified Hemocyanin Glycoprotein Solution, as follows: Hemocyanin Glycoprotein Solution was prepared by removing hemocytes and other debris from native lobster hemolymph. Hemocyanin glycoproteins remained in the resulting solution at physiologically normal concentrations. Approximate hemocyanin concentrations were measured and subsequent dilutions to achieve desired test concentrations were performed in a simple salt buffer to mimic ionic concentration of the hemolymph. Clarified Hemocyanin Glycoprotein Solution was prepared by further concentrating the hemocyanin proteins present in the Hemocyanin Glycoprotein Solution as previously described by placing the Hemocyanin Glycoprotein Solution into 10 kDa molecular weight cutoff protein concentrators and centrifuging until about half of the original volume remained in the top portion. The top portion of the solution was collected and approximate hemocyanin concentrations were measured. Subsequent dilutions to achieve desired test concentrations were performed by diluting the Clarified Hemocyanin Glycoprotein in the <10 kDa flowthrough as the dilutant.

### Cell Preparation and Viability

Standard preparation of HDFa and HaCaT cells was conducted. Human keratinocytes (HaCaT, 1×10⁴ cells per well) and adult human dermal fibroblasts (HDFa, 4×10⁴ cells per well) were seeded in black 96-well plates with complete cell culture media and cultured in humidified incubator at 37° C., 5% CO₂ until cells were confluent. A co-treatment of the various glycoprotein concentrations and DCFDA dye (50 μM) in serum free media was added to the HDFa cells for 1 hour, whereas for the HaCaTs media containing the test concentrations of glycoprotein samples were introduced into the cells first for 1 hour, followed by washing with PBS, and treatment with DCFDA dye (10 μM) prepared in Hanks Balanced Salt Solution (HBSS) for 45 minutes. A positive antioxidant control of 500 M ascorbic acid (vitamin C) was used for the HaCaT cells, while 250 M ascorbic acid (vitamin C) was used for the HDFa cells. The treatments were then removed, and cells were washed twice with PBS prior to the addition of hydrogen peroxide (1 mM HaCaT, 0.25 mM HDFa) prepared in HBSS. Fluorescence readings at Ex/Em 485/535 nm were recorded using a Varioskan Lux (Thermo Scientific) plate reader after 1 hour.

A cytotoxicity assay was run to determine appropriate dosage ranges for HDFa and HaCaT cells to maintain viability. Cells were dosed with resazurin reagent following kit instructions (Abcam Resazurin Assay Kit ab129732, abcam.com) and fluorescence was measured at 560 nm/590 nm using a plate reader. The resazurin assay is a fluorometric/colorimetric assay used to measure the metabolic capacity of live cells. It does so by measuring the reduction of oxidized non-fluorescent blue resazurin to a red fluorescent dye (resorufin) by the mitochondrial respiratory chain in live cells. The amount of resorufin produced is directly proportional to the number of living cells. Resorufin has Ex/Em of 530-560 nm/590 nm and absorbance at 570 nm.

### Antioxidant Assay

A DCFDA antioxidant assay was performed to measure the antioxidant capacity of Hemocyanin Glycoproteins. An Abeam DCFDA/H2DCFDA—Cellular ROS Assay Kit (Abeam Cellular ROS Assay Kit abab113851, abcam.com) was used following kit instructions. The assay measures antioxidant capacity by using the cell permeant reagent 2′,7′-dichlorofluorescein diacetate (DCFDA) to semi quantitatively assess reactive oxygen species (ROS) in live cell samples. Oxidization of DCFDA into the highly fluorescent 2′, 7′-dichlorofluorescein (DCF), can be measured by fluorescence spectroscopy with excitation/emission at 485 nm/535 nm.

HDFa cells were incubated with Hemocyanin Glycoproteins in a range of concentrations and 50 μM of DCFDA for 1 hour. Cells were then dosed with the pro-oxidant compound, hydrogen peroxide (H₂O₂) at a concentration of 250 μM to induce the production of free radicals and reactive oxygen species (ROS). 250 μM Ascorbic Acid (Vitamin C) was used as a positive control to compare the ROS-scavenging ability of the Hemocyanin Glycoprotein solutions. Approximate Hemocyanin Glycoprotein dosages followed a range of: 0.01 mg/ml, 0.1 mg/ml, 1 mg/ml, 10 mg/ml, 16 mg/ml and 25 mg/ml.

HaCaT cells were incubated with Hemocyanin Glycoprotein Solution in a range of concentrations and 10 μM of DCFDA for 1 hour. Cells were then dosed with the pro-oxidant compound, hydrogen peroxide (H₂O₂) at a concentration of 1 mM to induce the production of free radicals and reactive oxygen species (ROS). 500 μM Ascorbic Acid (Vitamin C) was used as a positive control to compare the ROS-scavenging ability of the Hemocyanin Glycoprotein solutions. Approximate Hemocyanin Glycoprotein dosages followed a range of: 0.01 mg/ml, 0.1 mg/ml, 1 mg/ml, 2 mg/ml, and 4 mg/ml.

### Results—Antioxidant Effect

The antioxidant assay demonstrated a significant decrease in reactive oxygen species (ROS) detected in both HDFa and HaCaT cells stimulated with the pro-oxidant compound, hydrogen peroxide (H₂O₂) that were treated with Hemocyanin Glycoproteins at concentrations of 0.1 mg/mL to 10 mg/ml, indicating strong antioxidant effects. In fact, in the fibroblast assay, the ROS-scavenging capacity of the Hemocyanin Glycoproteins at these concentrations was greater than that of the positive control, 250 μM Vitamin C. In the HaCaT keratinocyte assay, the effect measured in the 0.1 mg/ml Clarified Hemocyanin Glycoprotein Solution group was comparable to the 500 μM Vitamin C positive control. The strong antioxidant effect of the methods and compositions of the invention result in various skin benefits stemming from the antioxidant activity increase and reduction of free-radicals in the skin observable as one or more of reduction of signs of aging, reduced dullness, reduced wrinkles, reduced fine lines, reduced sensitivity to damage caused by free radicals, reduced sensitivity to environmental damage, reduced sensitivity to pollutants, promotion of skin radiance, improved complexion, new dermal skin cell growth, and a gentle exfoliating effect.

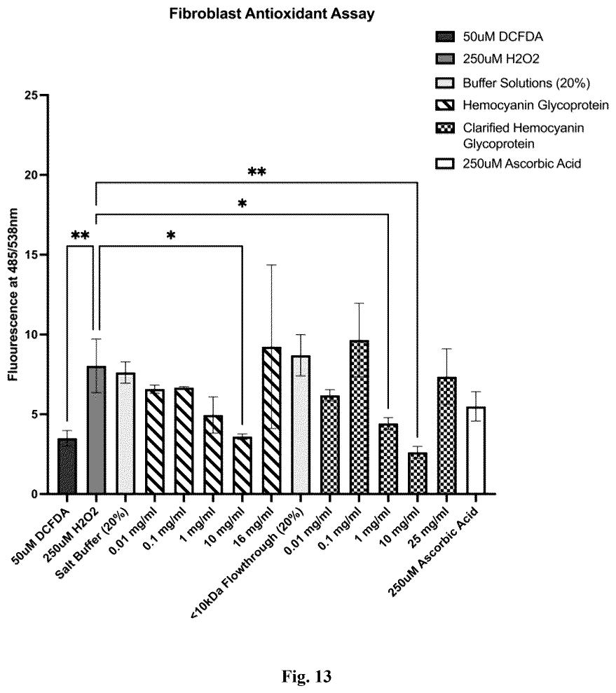

As shown in FIG. 13, in the Fibroblast Antioxidant Assay, treatment with 250 μM of H₂O₂ alone caused a significant increase in the level of ROS compared to the cells left untreated (**p adj.<0.01). Treatment with Hemocyanin Glycoprotein Solution at 10 mg/ml and with Clarified Hemocyanin Glycoprotein Solution at 1 mg/ml and 10 mg/ml resulted in significantly decreased ROS levels compared to the H₂O₂ group alone (*p adj.<0.05, **p adj.<0.01). Treatment with 250 μM of the known antioxidant, Ascorbic Acid (Vitamin C) resulted in a decrease in the mean level of ROS compared to the H₂O₂ alone, however, statistically significant reduction was not observed (n.s., p=0.11). It is unclear why 1 mg/ml of the Clarified Hemocyanin Glycoprotein Solution significantly decreased the levels of ROS, but 1 mg/ml of Hemocyanin Glycoprotein Solution did not, however, the mean was lower than the H₂O₂ group alone, but not in a statistically significant manner (1 mg/ml Hemocyanin Glycoprotein Solution mean=4.95 vs 250 μM H₂O₂ mean=8.03, p adj.=0.14). The antioxidant properties of the Hemocyanin Glycoprotein seem to follow a dose-dependent relationship, where concentrations above 10 mg/mL show reduced efficacy. The reason for this is unknown. However, overall, this data provides evidence of a strong anti-inflammatory effect of Hemocyanin Glycoproteins at the appropriate concentrations.

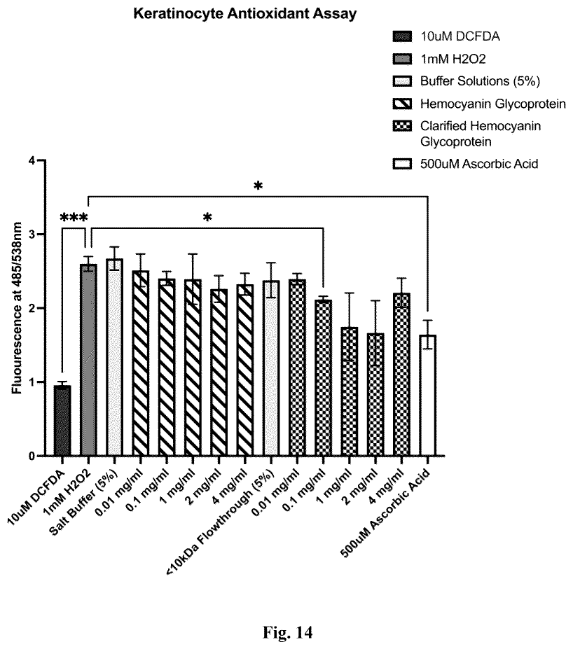

As shown in FIG. 14, in the Keratinocyte Antioxidant Assay, treatment with 1 mM of H₂O₂ alone caused a significant increase in the level of ROS compared to the cells left untreated (**p adj.<0.001). Treatment with Clarified Hemocyanin Glycoprotein Solution at 0.1 mg/mL resulted in significantly decreased ROS levels compared to the H₂O₂ group alone (*p adj.<0.05). Treatment with Clarified Hemocyanin Glycoprotein Solution at 1 mg/mL and 2 mg/ml also resulted in lower mean values (1 mM H₂O₂ mean=2.600 vs 1 mg/ml Clarified Hemocyanin Glycoprotein Solution mean=1.748 and 2 mg/mL mean=1.664), however, this reduction was not statistically significant (1 mg/ml: n.s., p adj.=0.34)(2 mg/ml: n.s., p adj.=0.27). Treatment with 500 μM of the known antioxidant, Ascorbic Acid (Vitamin C) resulted in a significant reduction in ROS levels compared to the H₂O₂ alone (*p adj.<0.05). It is unclear why the Concentrated Hemocyanin Glycoprotein Solution appeared to have an antioxidant effect, while the Hemocyanin Glycoprotein Solution did not. However, overall this data provides evidence of a strong antioxidant effect of Hemocyanin Glycoproteins at the appropriate concentrations.

### Example F

RNA Microarray: An RNA array performed on fibroblasts dosed with concentrated hemolymph, primarily containing lobster hemocyanin/glycoprotein, was conducted to measure modulation in inflammatory or immune-related gene expression, revealing clear anti-inflammatory effects.

### Method

Human primary fibroblasts (Thermofisher Scientific, catalog #C0135C) were treated with cell culture media containing 5% concentrated hemolymph (primarily, the Glycoprotein Hemocyanin) by volume for 24 hours. A salt buffer that matched the mineral content, pH and salinity levels of the hemolymph was used as a negative control. The results of the microarray were analyzed by fold change (FC) and p values for each individual gene in the array, as well as information such as z-scores on specified pathways of interest. The specific area of interest in this context was exploring the impact on inflammatory and inflammation-related pathways.

### Results

Overall, this data unveiled significant, novel immunomodulatory effects of lobster hemocyanin—demonstrating a clear anti-inflammatory effect via reduction of pro-inflammatory signaling pathways such as TNF, NF-kappa B, IL17 and chemokine signaling pathways, and downregulation of key markers including CXCL1, CXCL3, IL1b, MYD88 and NFKBIA. Separately and complementary, we have observed significant efficacy via test compositions comprising lobster hemocyanin as described in Example A (Soothing Hydration Cream, Marin Brands, Inc., Portland, Maine, U.S.A.), with inflammatory skin disorders such as eczema and psoriasis, which could be due to the downregulation of these pro-inflammatory pathways, resulting in reduction of pro-inflammatory cytokines and chemokines that contribute to the skin disease pathology. This data is the first of its kind in linking hemolymph with the ability to treat inflammatory skin conditions. Four pathways in the human fibroblasts that were significantly downregulated due to treatment with 5% hemolymph were chemokine signaling pathway (z-score −1.7. p<0.05), NF-kappa B signaling pathway (z-score −3.0, p<0.01), IL-17 signaling pathway (z-score −3.4, p<0.01), and INF signaling pathway (z-score −4.5, p<0.01) (Table 11).

Table 11—PAGE_Fibroblasts (2) selected inflammatory pathways of interest with significant lowered z-scores.

TABLE 11

| | Genes | | | | Genes | Genes |
|---|---|---|---|---|---|---|
| Name | (tot) | Z score | p-value | FDR | (up) | (down) |
| hsa04062_Chemokine_signaling_pathway | 178 | −1.7025 | 0.031968 | 0.17183 | 96 | 82 |
| hsa04064_NF-kappa_B_signaling pathway | 96 | −2.9893 | 0.001998 | 0.014319 | 40 | 56 |
| hsa04657_IL-17_signaling_pathway | 89 | −3.3792 | 0.000999 | 0.0085914 | 34 | 55 |
| hsa04668_TNF_signaling_pathway | 111 | −4.8492 | 0.000999 | 0.0085914 | 36 | 75 |

As shown in Table 12, heatmap data shows z-scores (PAGE Z-score scale −4 to 4) of pathways of interest with four inflammatory pathways chemokine signaling, NF-kappa B, IL-17, and TNF highlighted. All four have significantly reduced z-scores.

TABLE 12

| Fibroblast Pathway | PAGE Z-score |
|---|---|
| hsa04062_Chemokine_signaling_pathway | −2 |
| hsa040664_NF-kappa_B_signaling_pathway | −3 |
| hsa04657_IL-17_signaling_pathway | −3 |
| hsa04668_TNF_signaling_pathway | −4 |

TNF signaling pathway is activated by the proinflammatory cytokine, TNF (tumor necrosis factor). This pathway is critical for the immune response and must be tightly regulated. Overactivation and hyperactivity of this pathway can create a state of chronic inflammation that is known to contribute to the symptoms of skin disorders like eczema and psoriasis. Additionally, increasing levels of TNF found in serum and tissue have been found to directly correlate to the extent, duration, and activity of vitiligo. TNF also can activate other pro-inflammatory pathways, such as NFKB and IL-17. (Webster, J. D. and Domagoj, V, The Balance of TNF Mediated Pathways Regulates Inflammatory Cell Death Signaling in Health and Diseased Tissues, Front. Cell Dev. Biol, 20 May 2020 Vol. 8 2020, https://www.frontiersin.org/journals/cell-and-developmental-biology/articles/10.3389/fcell.2020.00365/full) (Pocino, K. et al, Tumor Necrosis Factor-Alpha: Ally and Enemy Protean Cutaneous Sceneries, Int J Mol Sci, 2024 Jul. 16; 25(14):7762, https://pmc.ncbi.nlm.nih.gov/articles/PMC11276697/#:~:text=The%20role%20of%20TNF%2D%CE%B1%20is%20overriding%20in%20the%20pathophysiology,plaques%20%5B2%2C12%5 D). NFKB (nuclear factor kappa-light-chain-enhancer of activated B cells) is a transcription factor that regulates the expression of several inflammatory genes, and is responsible for the increase of many pro-inflammatory cytokines and chemokines such as TNF, IL-1, IL-6, CXCL1, etc. This pathway is widely recognized as a pro-inflammatory pathway, and is involved in many inflammatory diseases, including ones that plague the skin (Liu, T, et al. NF-κB signaling in Inflammation, Signal Transduction and Targeted Therapy 2, Article number: 17023 (2017), https://www.nature.com/articles/sigtrans201723; Wullaert, A. et al, NF-κB in the regulation of epithelial homeostasis and inflammation, Cell Research 21, 146-158 (2011), https://www.nature.com/articles/cr2010175). IL17 and IL17-mediated genes have been identified to play a major role in inflammatory skin conditions like eczema psoriasis. In fact, IL17E (also called IL25), which is mediated by IL17, has been shown to cause skin inflammation, overproliferation of keratinocytes (a key mechanism in the development of psoriasis plaques), and production of inflammatory cytokines and chemokines. Inhibition of the IL17 pathway has been an effective therapeutic strategy for treating psoriasis and hidradenitis suppurativa, another chronic inflammatory skin condition (Huangfu, L, et al., The IL-17 family in diseases: from Bench to Bedside, Signal Transduction and Targeted Therapy 8, Article number: 402 (2023), https://www.nature.com/articles/s41392-023-01620-3 #ref-CR177). Chemokine signaling pathways are also important mediators of inflammation, as they are critical for directing cell migration and recruitment of immune cells to sites of infection. However, elevated levels or unregulated signaling of chemokines can cause excessive and persistent inflammation characteristic of autoimmune and chronic inflammatory diseases, including ones of the skin like psoriasis (Chemokine Signaling Pathways Poster, R&D Systems, Inc. (2019), https://www.rndsystems.com/pathways/chemokine-signaling-pathways). Beyond pathway analysis, when looking at the level of individual genes, treatment with 5% Hemocyanin caused a significant downregulation of several key markers of inflammation. Several chemokines, including CXCL1 and CXCL3 were downregulated (FC −1.2 and −0.4 respectively, p<0.01). Pro-inflammatory cytokines such as TNF and IL1b were also significantly downregulated (FC −0.2 and −0.5 respectively, p<0.01). Individual genes within inflammatory signaling pathways such as MYD88 and NFKBIA were also significantly downregulated (FC −0.6 and −1.2 respectively, p<0.01) (Table 13).

Table 13—Fold changes for a select number of important genetic markers of inflammation. List generated to include only genes with adjusted p values <0.01.

TABLE 13

| Gene Name | logFC | P.Value | Adj. P.Value | Unique ID | Test |
|---|---|---|---|---|---|
| NFKBIA | −1.24 | 0 | 0 | NFKBIA | Fibroblast |
| CXCL1 | −1.21 | 0 | 0 | CXCL1 | Fibroblast |
| MYD88 | −0.64 | 0 | 0 | MYD88 | Fibroblast |
| IL1B | −0.539 | 0 | 0 | IL1B | Fibroblast |
| CXCL3 | −0.448 | 0 | 0 | CXCL3 | Fibroblast |
| TNF | −0.166 | 0 | 0 | TNF | Fibroblast |

In conclusion, this data unveiling the immunomodulatory effects of lobster hemocyanin demonstrates a clear anti-inflammatory effect via reduction of pro-inflammatory signaling pathways such as TNF, NF-kappa B, IL17 and chemokine signaling pathways, and downregulation of key markers including CXCL1, CXCL3, IL1b, MYD88 and NFKBIA. Separately and complementary, significant efficacy was observed with test compositions containing lobster hemocyanin glycoprotein via (SOOTHING HYDRATION CREAM, Marin Brands, Inc., Portland, Maine, U.S.A.), with inflammatory skin disorders such as eczema and psoriasis, which could be due to the downregulation of these pro-inflammatory pathways, resulting in reduction of pro-inflammatory cytokines and chemokines that contribute to the skin disease pathology. This data is the first of its kind in linking hemolymph with the ability to treat inflammatory skin conditions, as well as normalization of proliferation in non-neoplastic, overproliferative disorder such as that of keratinocytes, of particular interest in the context of psoriasis.

The use of the terms “a,” “an,” “the,” and similar referents in the context of describing the presently claimed invention (especially in the context of the claims) are to be construed to cover both the singular and the plural, unless otherwise indicated herein or clearly contracted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments the values may range in value either above or below the stated value in a range of approximately ±5%; in other embodiments the values may range in value either above or below the stated value in a range of approximately ±2%; in other embodiments the values may range in value either above or below the stated value in a range of approximately ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entireties. The present invention may be embodied in other specific forms without departing from the spirit or essential attributes thereof and, accordingly, reference should be made to the appended claims, rather than to the foregoing specification, as indicating the scope of the invention.

We claim:

1. A method for treating atopic dermatitis in a mammal, comprising the steps of:
(a) providing a composition consisting essentially of hemocyanin derived from organisms selected from the group consisting of:
lobster species selected from Nephrops novegicus, Homarus gammarus, or Panularis elephas, or Homarus americanus,
crab species selected from Carcinus maenas, Portunus pelagicus, Portunus trituberculatus, Callinectes sapidus, Cancer pagurus, Metacarcinus magister, or Chionoecetes;
crawfish species selected from Procambarus clarkii or Procambarus zonangulus; and
shrimp species selected from Penaeus monodon, Pandalus borealis, Acetes japonicas, or Trachysalambria curvirostris;
wherein hemocyanin is from about 1% to less than about 20% by weight based on the total weight of the composition; and
(b) administering the composition to the mammal;
whereby symptoms of atopic dermatitis selected from eczema, perioral dermatitis, contact dermatitis, seborrheic dermatitis, radiation-induced dermatitis, chemotherapy-induced dermatitis, and topical steroid use-induced dermatitis of the mammal are improved.

2. The method of claim 1 wherein hemocyanin has a concentration from about 6 mg/mL to about 10 mg/mL.

3. The method of claim 1 wherein hemocyanin is administered in a mass dosage of from about 3 mg to about 30 mg.

4. The method of claim 1, wherein the hemocyanin is a recombinant protein or peptide.

5. The method of claim 4, wherein the recombinant hemocyanin protein shares at least 85% sequence identity compared to a wildtype hemocyanin protein or peptide.

6. The method of claim 1 wherein skin texture, skin smoothness, skin softness, skin moisturization, and overall look and feel are improved, and skin dryness, skin flakiness and scaling, overall skin irritation, overall skin discomfort, itchiness, redness, crusting, blotching, erythema, and hyperpigmentation are reduced.

7. The method of claim 1, wherein the composition is a topical composition administered topically to the skin daily for up to about 2 days to about 4 weeks.

8. The method of claim 7, wherein the composition is a topical composition administered topically to the skin daily for up to about 1 week.

9. The method of claim 1, wherein the composition contains a dosage of hemocyanin from about 1 ng to about 500 mg.

10. A method for treating skin inflammation conditions in a mammal, comprising the steps of:
(a) providing a composition consisting essentially of hemocyanin derived from organisms selected from the group consisting of:
lobster species selected from Nephrops novegicus, Homarus gammarus, or Panularis elephas, or Homarus americanus,
crab species selected from Carcinus maenas, Portunus pelagicus, Portunus trituberculatus, Callinectes sapidus, Cancer pagurus, Metacarcinus magister, or Chionoecetes;
crawfish species selected from Procambarus clarkii or Procambarus zonangulus; and
shrimp species selected from Penaeus monodon, Pandalus borealis, Acetes japonicas, or Trachysalambria curvirostris;
wherein hemocyanin is from about 1% to about 25% by weight based on the total weight of the composition; and
(b) administering the composition to the mammal;
whereby symptoms of inflammation of the skin of the mammal are improved.

11. The method of claim 10 wherein hemocyanin has a concentration from about 0.1 mg/mL to about 25 mg/mL.

12. The method of claim 10 wherein hemocyanin is administered in a mass dosage of from about 0.1 mg to about 25 mg.

13. The method of claim 10, wherein the hemocyanin protein is a recombinant protein or peptide.

14. The method of claim 13, wherein the recombinant hemocyanin protein shares at least 85% sequence identity compared to a wildtype hemocyanin protein or peptide.

15. The method of claim 10 wherein inflammation is reduced by downregulation or reduction of pro-inflammatory signaling pathways.

16. The method of claim 15 wherein the inflammation skin condition is a disorder selected from eczema, psoriasis, rosacea, acne, hyperpigmentation, itching, and rashes.

17. The method of claim 10, wherein the composition is a topical composition administered topically to the skin daily for up to about 2 days to about 4 weeks.

18. The method of claim 17, wherein the composition is a topical composition administered topically to the skin daily for up to about 1 week.

19. The method of claim 10, wherein the composition contains a dosage of hemocyanin from about 1 ng to about 500 mg.

20. A method for increasing hyaluronic acid and collagen production by fibroblasts in the skin of a mammal, comprising the steps of:
(a) providing a composition consisting essentially of hemocyanin derived from organisms selected from the group consisting of:
lobster species selected from Nephrops novegicus, Homarus gammarus, or Panularis elephas, or Homarus americanus,
crab species selected from Carcinus maenas, Portunus pelagicus, Portunus trituberculatus, Callinectes sapidus, Cancer pagurus, Metacarcinus magister, or Chionoecetes;
crawfish species selected from Procambarus clarkii or Procambarus zonangulus; and
shrimp species selected from Penaeus monodon, Pandalus borealis, Acetes japonicas, or Trachysalambria curvirostris;
wherein hemocyanin is from about 1% to less than about 20% by weight based on the total weight of the composition; and
(b) administering the composition to the mammal;
whereby fibroblasts in the skin of a mammal increase production of hyaluronic acid and collagen thereby symptoms of skin conditions of the mammal are improved.

21. The method of claim 20 wherein hemocyanin has a concentration from about 0.3 mg/mL to about 6 mg/mL.

22. The method of claim 20 wherein hemocyanin is administered in a mass dosage of from about 0.05 mg to about 3 mg.

23. The method of claim 20, wherein the hemocyanin protein is a recombinant protein or peptide.

24. The method of claim 23, wherein the recombinant hemocyanin protein shares at least 85% sequence identity compared to a wildtype hemocyanin protein or peptide.

25. The method of claim 20 wherein skin texture, skin smoothness, skin softness, skin moisturization, and overall look and feel are improved, and skin dryness, skin flakiness and scaling, overall skin irritation, overall skin discomfort, itchiness and redness are reduced.

26. The method of claim 20, wherein the composition is a topical composition administered topically to the skin daily for up to about 2 days to about 4 weeks.

27. The method of claim 26, wherein the composition is a topical composition administered topically to the skin daily for up to about 1 week.

28. The method of claim 20, wherein the composition contains a dosage of hemocyanin from about 1 ng to about 500 mg.

29. A topical composition for treating conditions of the skin comprising hemocyanin derived from marine organisms selected from the group consisting of: lobster species selected from Nephrops novegicus, Homarus gammarus, or Panularis elephas, or Homarus americanus; crab species selected from Carcinus maenas, Portunus pelagicus, Portunus trituberculatus, Callinectes sapidus, Cancer pagurus, Metacarcinus magister, or Chionoecetes; crawfish species selected from Procambarus clarkii or Procambarus zonangulus; and shrimp species selected from Penaeus monodon, Pandalus borealis, Acetes japonicas, or Trachysalambria curvirostris; wherein hemocyanin is from about 1% to about 25% by weight based on the total weight of the composition.

30. The topical composition of claim 29 wherein the hemocyanin has a concentration of from about 0.1 mg/mL to about 25 mg/mL.

31. The topical composition of claim 29 wherein the hemocyanin has a mass dosage of from about 0.1 mg to about 30 mg.

32. The composition of claim 29 further comprising water, Butyrospermum parkii shea butter fruit, cetyl alcohol, glyceryl stearate, glycerin, caprylic/capric triglyceride, sorbitan oleate decylglucoside crosspolymer, polyacrylamide, C13-14 isoparaffin, Laureth-7, Cocos nucifera (coconut) oil, squalane, tocopherol (vitamin E), hyaluronic acid, sodium benzoate, sodium levulinate, and sodium phytate, or alternatively polyisobutene, caprylic/capric triglyceride, squalane, synthetic beeswax, 12-hydroxystearic acid, Euphorbia cerifera (Candelilla) wax, glyceryl adipate, Glyceryl Stearate, tocopherol, stevia, and optionally flavor.

33. A method for increasing antioxidant activity in the skin of a mammal, comprising the steps of:
(a) providing a composition consisting essentially of hemocyanin derived from organisms selected from the group consisting of:
lobster species selected from Nephrops novegicus, Homarus gammarus, or Panularis elephas, or Homarus americanus,
crab species selected from Carcinus maenas, Portunus pelagicus, Portunus trituberculatus, Callinectes sapidus, Cancer pagurus, Metacarcinus magister, or Chionoecetes;
crawfish species selected from Procambarus clarkii or Procambarus zonangulus; and
shrimp species selected from Penaeus monodon, Pandalus borealis, Acetes japonicas, or Trachysalambria curvirostris;
wherein hemocyanin is from about 1% to about 25% by weight based on the total weight of the composition; and
(b) administering the composition to the mammal;
whereby antioxidant activity on the skin of the mammal is increased and free radicals are reduced.

34. The method of claim 33 wherein hemocyanin has a concentration from about 0.1 mg/mL to about 25 mg/mL.

35. The method of claim 33 wherein hemocyanin is administered in a mass dosage of from about 0.1 mg to about 25 mg.

36. The method of claim 33, wherein the hemocyanin protein is a recombinant protein or peptide.

37. The method of claim 36, wherein the recombinant hemocyanin protein shares at least 85% sequence identity compared to a wildtype hemocyanin protein or peptide.

38. The method of claim 33, wherein the composition is a topical composition administered topically to the skin daily for up to about 2 days to about 4 weeks.

39. The method of claim 38, wherein the composition is a topical composition administered topically to the skin daily for up to about 1 week.

40. The method of claim 33, wherein the composition contains a dosage of hemocyanin from about 1 ng to about 500 mg.

41. The method of claim 33, wherein the antioxidant activity increase and reduction of free-radicals results in one or more of reduction of signs of aging, reduced dullness, reduced wrinkles, reduced fine lines, reduced sensitivity to damage caused by free radicals, reduced sensitivity to environmental damage, reduced sensitivity to pollutants, promotion of skin radiance, improved complexion, new dermal skin cell growth, and a gentle exfoliating effect.
